(19) United States
(12) Patent Application Publication
(10) Pub. No.: US 2009/0202638 A2
(43) Pub. Date: Aug. 13, 2009

(54) BMP GENE AND FUSION PROTEIN

(75) Inventors: Chisa Hidaka, New York, NY (US); Wei Zhu, Kew Gardens, NY (US)

Correspondence Address: DARBY & DARBY P.C., P.O. BOX 770, Church Street Station, New York, NY, 10008-0770, UNITED STATES, 212-527-7700, 212-527-7701, patent@darbylaw.com

(73) Assignee: New York Society for the Ruptured and Crippled Maintaining The Hospital for Special Surgery, New York, NY (US)

(21) Appl. No.: 11/577,112

(22) Filed: Jan. 28, 2008

Related U.S. Application Data
(60) Provisional application No. 60/622,490, filed on Oct. 27, 2004.

(51) Int. Cl. A61K 9/00; C07K 5/117; C07K 5/08; C07D 233/64; C07D 403/06; G06F 19/00; G06F 17/50; C12N 5/06; A61P 37/02; C12Q 1/02; A61K 38/12; A61K 38/07; A61K 31/53; A61K 39/00; A61P 35/00; A61K 35/12

(52) U.S. Cl. 424/484; 530/330; 530/331; 548/338.1; 548/314.4; 702/19; 703/1; 435/375; 514/11; 514/18; 514/245; 424/184.1; 424/185.1; 424/93.7; 435/29

(57) ABSTRACT

This invention relates to BMP fusion genes, BMP fusion proteins, and methods for making BMP fusion genes and BMP fusion proteins. The invention further relates to methods for treatment using BMP fusion genes and BMP fusion proteins. Additionally, the invention relates to BMP fusion gene and BMP fusion protein pharmaceutical compositions.

This application is a 371 National Phase of International Application No. PCT/US2005/038885, filed Oct. 26, 2005, which claims priority to U.S. provisional patent Application No. 60/622,490, filed Oct. 27, 2004. The contents of these two applications are hereby incorporated by reference in their entirety.

### FIELD OF THE INVENTION

This invention relates to a gene encoding a bone morphogenetic protein fusion protein (“BMP fusion gene”), a BMP fusion protein, methods for producing a BMP fusion protein, and methods for treatment using a BMP fusion gene or a BMP fusion protein.

### BACKGROUND

Bone morphogenetic proteins (BMPs) are proteins, which induce bone formation. BMPs are members of the transforming growth factor beta (TGF-β) superfamily of dimeric, disulfide-linked growth factors (Sampath, et al., J Biol. Chem. 1990; 265:13198-13205). BMP2 and BMP7 (also known as osteogenic protein-1) were initially co-purified from bovine bone. Two or more BMP genes are often co-expressed, for example, co-localization of BMP2 and BMP7 transcripts have been demonstrated in developing limbs of mouse embryos (Lyons et al., Mech Dev. 1995; 50:71-83). Other studies of embryogenesis have also shown the co-expression of several pairs of BMP genes is required for normal bone development (Katagiri et al., Dev Genet. 1998; 22:340-348; Solloway et al., Development. 1999; 126:1753-1768). In vitro studies suggest that co-expression of BMPs can result in the expression of heterodimeric BMPs (Aono et al., Biochem Biophys Res Commun. 1995; 210:670-677; Hazama et al., Biochem Biophys Res Commun. 1995; 209:859-866; Israel et al., Growth Factors. 1996; 13:291-300). However, “native” BMP heterodimers have not been isolated in vivo. Osteoblastic differentiation and ectopic bone formation studies have shown that BMP heterodimers are more potent than their respective homodimers (Aono et al.; Hazama et al.; Israel, et al.).

Spine fusion surgery is a commonly performed orthopedic procedure, which requires the formation of new bone around the spine to increase its stability. Non-union, or the failure of new bone to form, is a major complication of spine fusion, which occurs in up to 26% of posterolateral lumbar spine fusion cases (Steinman et al., Clin Ortho. 1992; 284:80-90; Rawlings et al., Spine. 1994; 8:563-571; Kimura et al., J Spinal Disord. 2001; 14:301-310). Autogenous bone grafting is the gold standard for induction of a spine fusion, but harvesting of the bone graft (typically from the iliac crest) is associated with significant morbidity in up to 30% of patients (Steinman et al.; Rawlings et al.; Kimura et al.; Arrington et al., Clin Ortho. 1996; 329:300-309). As such, the development of graft alternatives such as BMPs is of great interest. Recombinant BMP (rhBMP) homodimers have been shown to enhance spine fusion in animals and humans, but the doses required are extremely high (Celeste et al., Proc Ntl Aca Sci USA. 1990; 87:9843-9847; Boden et al., Spine. 2002; 27:2662-2673; Cook S D, Orthopedics. 1999; 22:669-671; Friedlander G E, J Bone Joint Surg Am. 2001; 83-A Suppl 1(Pt 2):S160-161; Sandhu et al., Spine. 2002; 27(16 Suppl 1):S32-38; Schmitt et al., J Ortho Res. 1999; 17:269-278; Wozney J M, Spine. 2002; 27(16 Suppl 1):S2-8; Zlotolow et al., J Am Acad Orth Surg. 2000; 8:3-9). Recombinant BMP2 (InFUSE, available from Medtronic) is supplied as a powder, which is mixed with sterile water and applied to an absorbable collagen sponge (available from Integra) prior to its topical application (i.e., direct application to the bone or to the vicinity of the bone). Recombinant BMP7 has been used in combination with a collagen carrier (the combination is marketed as OP-1 Implant, available from Stryker) to induce bone formation. OP-1 Implant is wetted to form a paste that is surgically implanted in a bone fracture gap.

Currently available rhBMPs are homodimers with two identical monomers linked by a disulfide bond. Post-translational processing of homodimer BMP proteins requires dimer formation followed by cleavage of the pro-proteins. Relative to homodimers, heterodimeric BMPs are more potent inducers of osteoblastic differentiation in vitro and enhancers of bone formation in vivo. BMP heterodimers are produced by co-transfection of target cells with two different BMP genes, which results in the production of both heterodimers and a mixture of homodimers. Because the monomers BMP2 and BMP7 are similar, BMP 2/7 heterodimers are difficult to purify from BMP2 homodimers, BMP7 homodimers, or a mixture thereof (Wozney J M. Mol Repro Devel. 1992; 32:160-167; Celeste et al., Proc Natl Acad Sci USA 1990; 87:9843-9847.

### SUMMARY OF THE INVENTION

The present invention provides a gene encoding two different BMP proteins (“BMP fusion gene”) in tandem, which results in expression of a BMP fusion protein (e.g., a BMP-2/7 fusion protein). A BMP fusion gene according to the present invention results in the expression of a single chain polypeptide, which contains both “halves” of a BMP heterodimer and forms by folding rather than dimerization. The BMP fusion gene and the BMP fusion protein of the present invention provide a BMP fusion protein equipotent to heterodimeric BMP. A BMP fusion gene of the present invention comprises a first BMP gene, a linker, and a second, different BMP gene, wherein the linker replaces the first BMP gene stop codon; the second, different BMP start codon; and the second, different BMP signal peptide nucleotide sequence. A preferred linker is comprised of about 60 base pairs (“bp”). An especially preferred linker encodes the amino acid sequence (Gly₄Ser)₄. A preferred BMP fusion gene is a human BMP fusion gene.

The present invention also provides a BMP fusion gene encoding a BMP protein component, a linker, and a nucleotide sequence encoding a TGF-β superfamily protein component, wherein the TGF-β superfamily protein component is different than the BMP protein component. Further, the invention provides a BMP fusion gene encoding a BMP-7/GDF-7; BMP-15/GDF-9; BMP-2/TGF-β1 or BMP-4/TGF-β1 fusion protein. An embodiment of the present invention provides a gene encoding BMP2 and BMP7 in tandem, which results in expression of a BMP2/7 fusion protein (i.e., a “BMP-2/7 fusion gene”). A BMP-2/7 fusion gene according to the present invention results in the expression of a single chain polypeptide, which contains both “halves” of a BMP-2/7 heterodimer and forms by folding rather than dimerization. The BMP-2/7 fusion gene and the BMP-2/7 fusion protein of the present invention provide a BMP-2/7 fusion protein equipotent to heterodimeric BMP-2/7. A BMP-2/7 fusion gene of the present invention comprises a BMP2 gene, a linker, and a BMP7 gene, wherein the linker replaces the BMP2 stop codon, the BMP7 start codon, and the BMP7 signal peptide nucleotide sequence. A preferred linker is comprised of about 60 base pairs (“bp”). An especially preferred linker encodes the amino acid sequence (Gly₄Ser)₄. A preferred BMP-2/7 fusion gene is a human BMP-2/7 fusion gene.

In an aspect of the present invention, a BMP fusion protein includes a first BMP protein component, a linker, and a second, different BMP protein component. A preferred BMP fusion protein is a human BMP fusion protein. A preferred linker is comprised of about 20 amino acids. An especially preferred linker is the amino acid sequence (Gly₄Ser)₄.

In another aspect of the present invention, a BMP fusion protein includes a BMP protein component, a linker, and a nucleotide sequence encoding a TGF-β superfamily protein component, wherein the TGF-β superfamily protein component is different than the BMP protein component. Further, according to the present invention, a BMP fusion protein is a BMP-7/GDF-7; BMP-15/GDF-9; BMP-2/TGF-β1 or BMP-4/TGF-β1 fusion protein.

According to the present invention, a BMP fusion protein comprises:

(a) a first BMP amino acid sequence as set forth in any one of SEQ ID NOS:2, 4 or 10 to 64;

(b) a linker as set forth in SEQ ID NO:5; and

(c) a second, different BMP amino acid sequence as set forth in any one of SEQ ID NOS:2, 4 or 10 to 64;

wherein the BMP amino acid sequence of (a) is different than the BMP amino acid sequence of (b) and either (a) or (b) is a BMP amino acid sequence as set forth in any one of SEQ ID NOs:2, 4 or 10 to 39.

Recombinant nucleic acids according to the present invention provide for efficient expression of BMP fusion gene constructs. Also encompassed are expression vectors in which the BMP fusion gene is operably associated with an expression control sequence. The invention extends to host cells transfected or transformed with the BMP fusion gene expression vector. The BMP fusion protein can be produced by isolating it from host cells grown under conditions that permit expression of the construct.

The methods of making a BMP fusion protein according to the present invention provide significant advantages over known methods of heterodimeric BMP production because a preparation is produced free of BMP homodimers, thus avoiding difficult, time-consuming and expensive separation of BMP heterodimers from BMP homodimers. Moreover, because of its increased potency, a BMP fusion protein can be administered in lower doses relative to BMP homodimers.

In one aspect, the present invention provides a method for producing a recombinant BMP fusion protein having bone stimulating activity comprising culturing a host cell containing a nucleotide sequence encoding BMP gene, and isolating the biologically active fusion protein from the culture medium.

Further, according to methods of the present invention, the BMP fusion gene or the BMP fusion protein can be administered to a patient to induce local or systemic bone formation.

A BMP-2/7 fusion protein of the present invention comprises a BMP2 protein component, a linker, and a BMP7 protein component. A preferred BMP-2/7 fusion protein is a human BMP-2/7 fusion protein. A preferred linker is comprised of about 20 amino acids. An especially preferred linker is the amino acid sequence (Gly₄Ser)₄.

In another aspect of the present invention, a BMP-2/7 fusion protein comprises:

(a) a BMP2 amino acid sequence as set forth in SEQ ID NO:2;

(b) a linker as set forth in SEQ ID NO:5; and

(c) a BMP7 amino acid sequence as set forth in SEQ ID NO:4.

Recombinant nucleic acids according to the present invention provide for efficient expression of BMP-2/7 fusion gene constructs. Also encompassed are expression vectors in which the BMP-2/7 fusion gene is operably associated with an expression control sequence. The invention extends to host cells transfected or transformed with the BMP-2/7 gene expression vector. The BMP-2/7 fusion protein can be produced by isolating it from the host cells grown under conditions that permit expression of the construct.

The methods of making a BMP-2/7 fusion protein according to the present invention provide significant advantages over known methods of heterodimeric BMP-2/7 production because a preparation is produced free of BMP homodimers, thus avoiding difficult, time-consuming and expensive separation of BMP heterodimers from BMP homodimers. Moreover, because of its increased potency, a BMP-2/7 fusion protein can be administered in lower doses relative to BMP homodimers.

In one aspect, the present invention provides a method for producing a recombinant BMP-2/7 fusion protein having bone stimulating activity comprising culturing a host cell containing a nucleotide sequence encoding BMP-2/7 gene, and isolating the biologically active fusion protein from the culture medium.

Further, according to methods of the present invention, the BMP-2/7 fusion gene or the BMP-2/7 fusion protein can be administered to a patient to induce local or systemic bone formation.

### BRIEF DESCRIPTION OF THE FIGURES

FIG. 1 shows the mRNA sequence (SEQ ID NO:1) (Genbank Accession #M22489) and amino acid sequence (SEQ ID NO: 2) of human BMP2. The stop codon of BMP2 is underlined and bold. Forward and reverse PCR primer sequences are shaded.

FIG. 2 shows the mRNA sequence (SEQ ID NO: 3) (Genbank Accession #X51801) and amino acid sequence (SEQ ID NO: 4) of human BMP7. The start codon and signal peptide nucleotide sequence of BMP7 are underlined and bold. Forward and reverse PCR primer sequences are shaded.

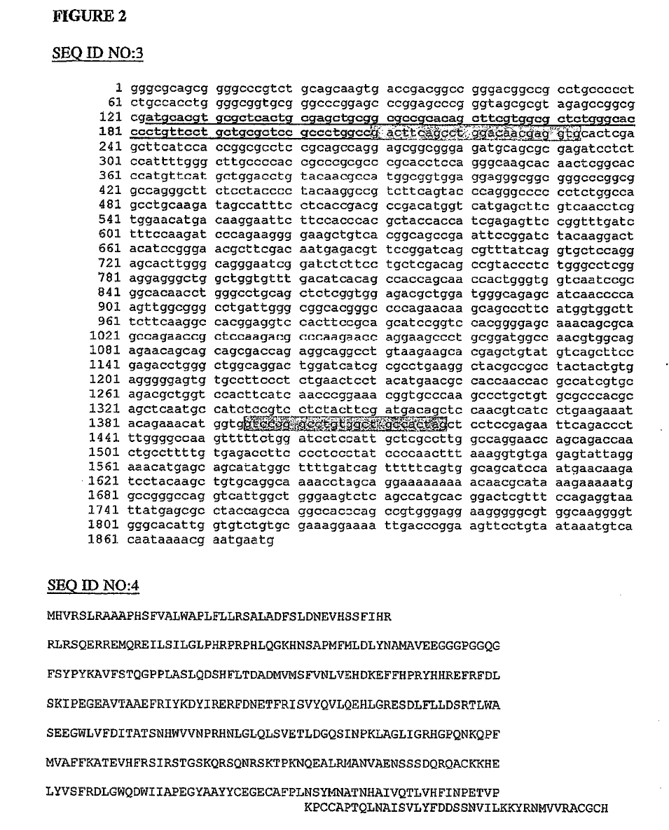

FIG. 3 illustrates construction of a BMP-2/7 fusion gene using serial PCR reactions.

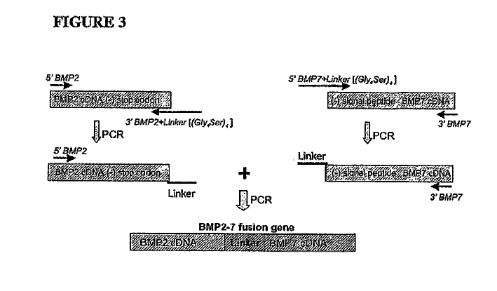

FIG. 4 depicts a graph, which shows BMP7 content in supernatants of cells transfected with pShuttleCMV-BMP-2/7, pCMV-GFP or medium only following immunoprecipitation with anti-BMP2 antibody.

FIG. 5 depicts graphs, which show OCN expression in C2C12 cells stimulated by A549 cell supernatants containing BMP-217 fusion protein (FIG. 5a) or BMP-2/7 heterodimer generated by co-transfection with BMP2 and BMP7 genes (FIG. 5b). FIG. 5a also shows that BMP-2/7 fusion protein at 2 ng/ml (1:5 dilution) resulted in an OCN level comparable to 1000 ng/ml of rhBMP2 or rhBMP7.

FIG. 6 depicts a graph, which shows OCN levels induced by maximal doses (i.e., about 1000 ng/ml) of rhBMP2 or rhBMP7.

FIG. 7 shows the amino acid sequence of human BMP3 precursor (SEQ ID NO:10, Genbank Accession #NP_{—}001192) and human BMP3A precursor (SEQ ID NO:11, Genbank Accession #P12645).

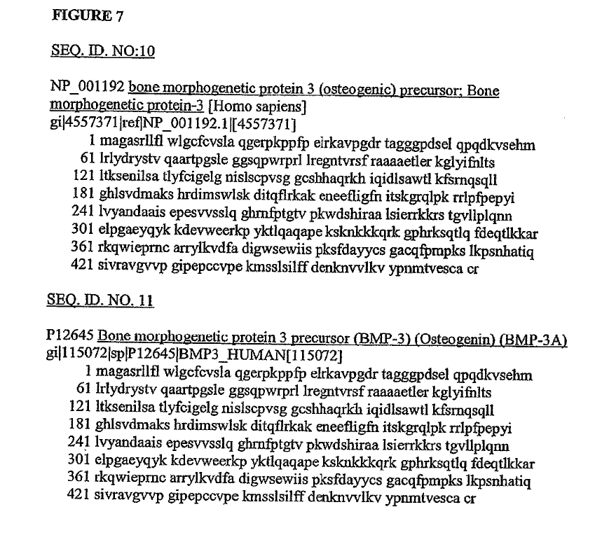

FIG. 8 shows the amino acid sequence of human BMP3B precursor (SEQ ID NO:12, Genbank Accession #P55107) and human BMP3B (SEQ ID NO:13, Genbank Accession #BAA08453).

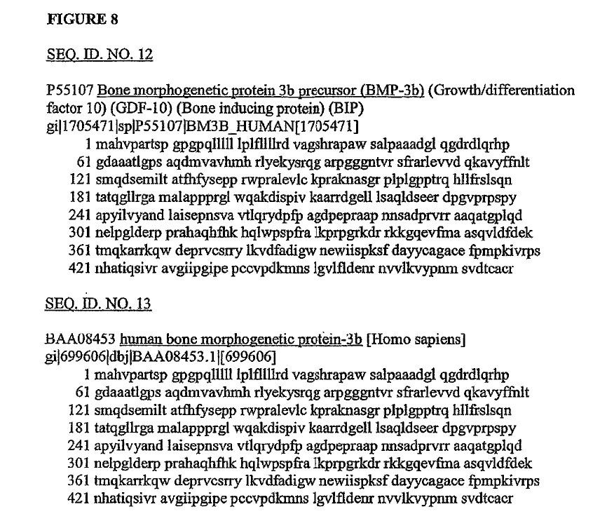

FIG. 9 shows the amino acid sequence of human BMP3B (SEQ ID NO:14, Genbank Accession #BAA008452, and SEQ ID NO:15, Genbank Accession #NP_{—}004953).

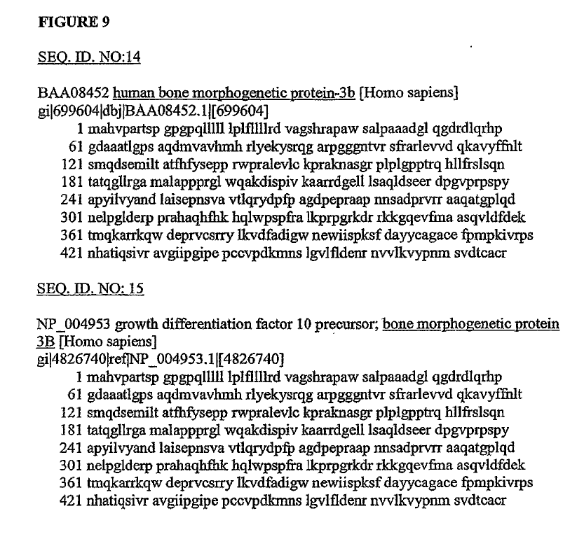

FIG. 10 shows the amino acid sequence of human BMP4 precursor (SEQ ID NO:16, Genbank Accession #P12644) and human BMP4 preprotein (SEQ ID NO:17, Genbank Accession #NP_{—}001193).

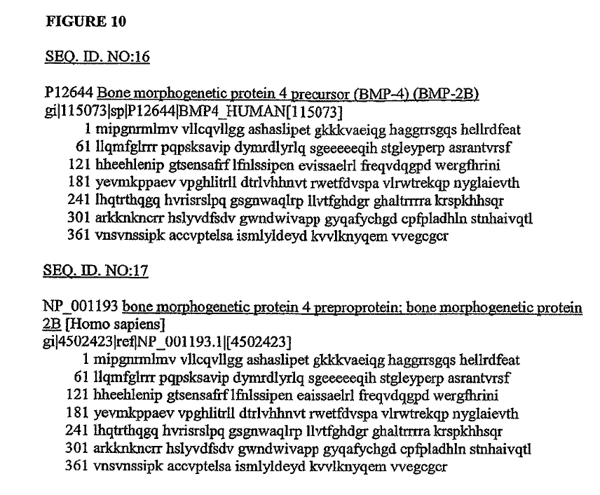

FIG. 11 shows the amino acid sequence of human BMP4 preprotein (SEQ ID NO:18, Genbank Accession #NP_{—}570911, and SEQ ID NO:19, Genbank Accession #NP_{—}570912).

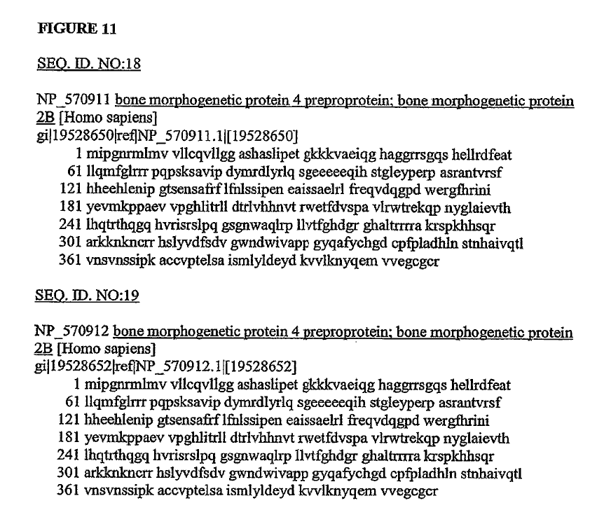

FIG. 12 shows the amino acid sequence of human BMP4 (SEQ ID NO:20, Genbank Accession #BAA06410, and SEQ ID NO:21, Genbank. Accession #AAC72278).

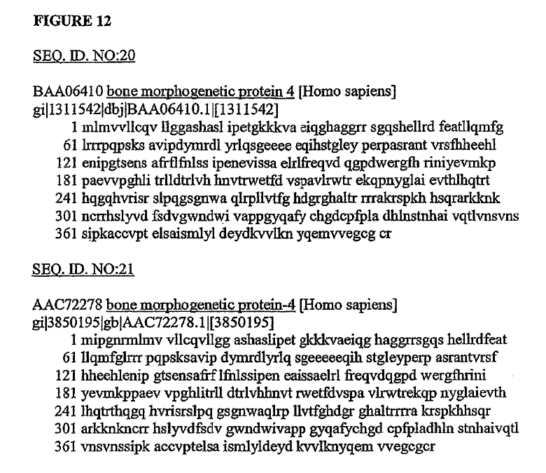

FIG. 13 shows the amino acid sequence of human BMP5 preprotein (SEQ ID NO:22, Genbank Accession #NP_{—}066551) and human BMP5 precursor (SEQ ID NO:23, Genbank Accession #P22003).

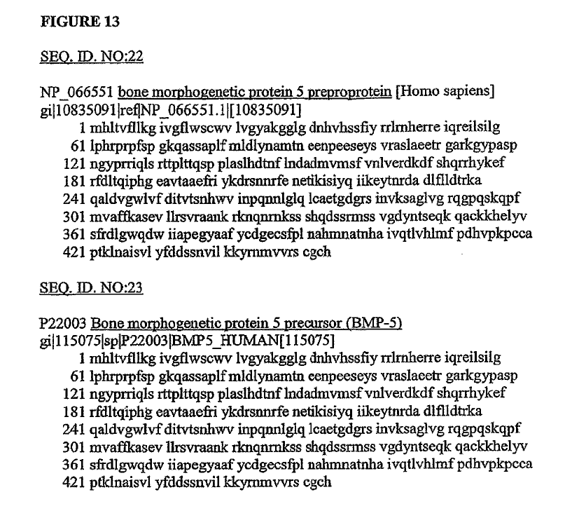

FIG. 14 shows the amino acid sequence of human BMP6 precursor (SEQ ID NO:24, Genbank Accession #P22004, and SEQ ID NO:25, Genbank Accession #NP_{—}001709).

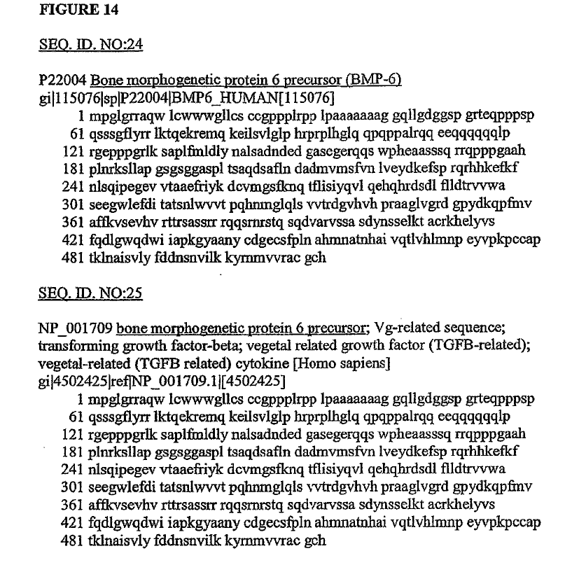

FIG. 15 shows the amino acid sequence of human BMP8B preprotein (SEQ ID NO:26, Genbank Accession #NP_{—}001711) and human BMP8B (SEQ ID NO:27, Genbank Accession #P34820).

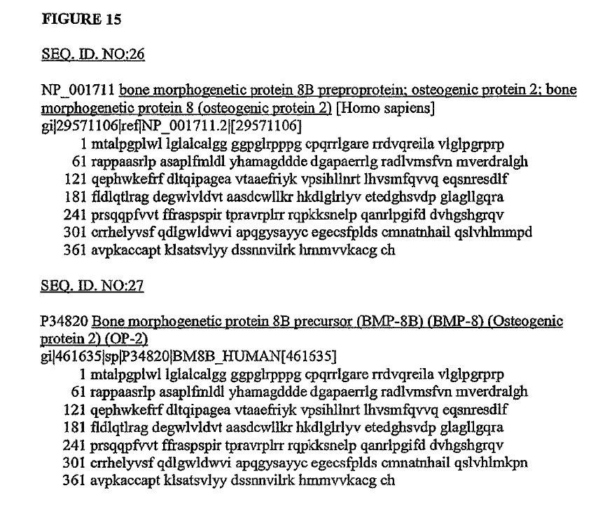

FIG. 16 shows the amino acid sequence of human BMP9 (SEQ ID NO:28, Genbank Accession #Q9UK05, and SEQ ID NO:29, Genbank Accession #NP_{—}057288).

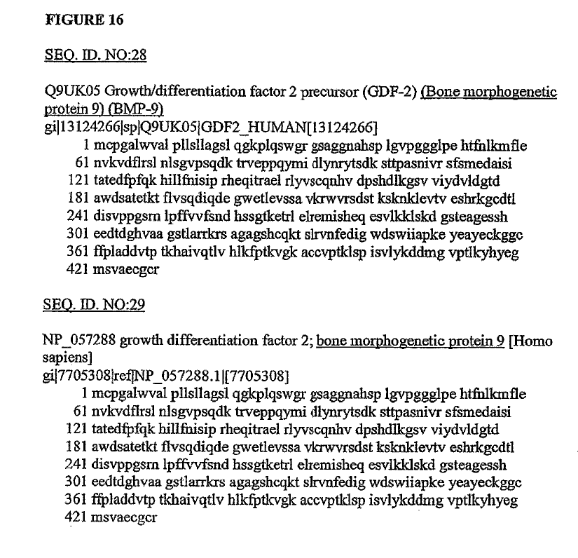

FIG. 17 shows the amino acid sequence of human BMP10 preprotein (SEQ ID NO:30, Genbank Accession #NP_{—}055297) and human BMP10 precursor (SEQ ID NO:31, Genbank Accession #O95393).

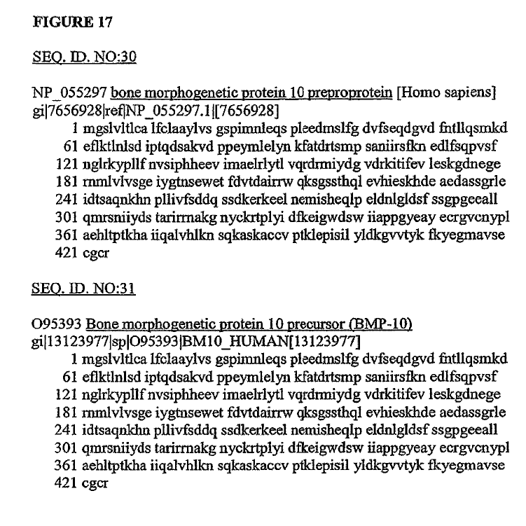

FIG. 18 shows the amino acid sequence of human BMP10 (SEQ ID NO:32, Genbank Accession #AAC77462) and human BMP11 (SEQ ID NO:33, Genbank Accession #AAC72852).

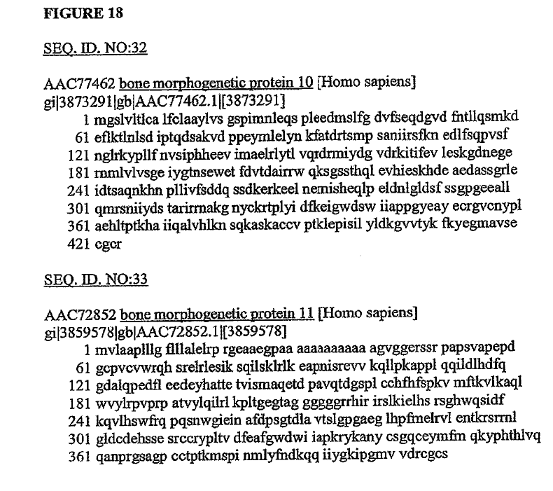

FIG. 19 shows the amino acid sequence of human BMP11 (SEQ ID NO:34, Genbank Accession #NP_{—}005802, and SEQ ID NO:35, Genbank Accession #O95390).

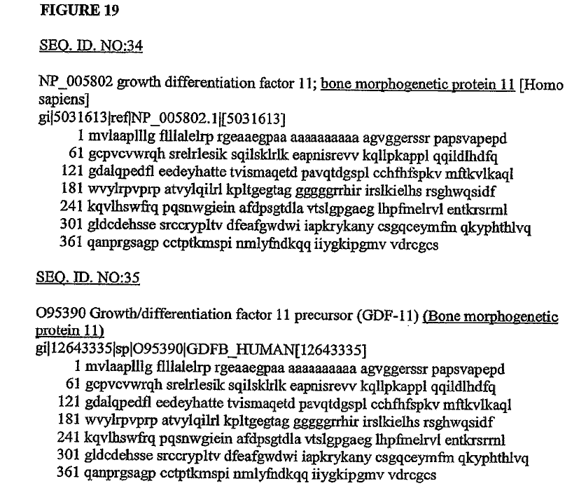

FIG. 20 shows the amino acid sequence of human BMP15 precursor (SEQ ID NO:36, Genbank Accession #O95972, and SEQ ID NO:37, Genbank Accession #NP_{—}005439).

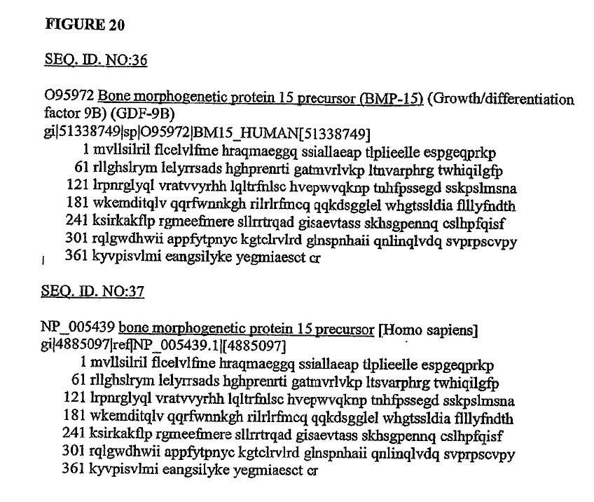

FIG. 21 shows the amino acid sequence of human TGFβ BMP (SEQ ID NO:38, Genbank Accession #AAA36737) and human BMPY (SEQ ID NO:39, Genbank. Accession #AAF15295).

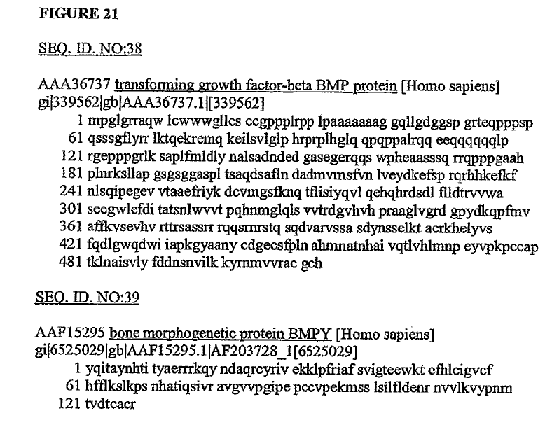

FIG. 22 shows the amino acid sequence of human embryonic GDF1 precursor (SEQ ID NO:40, Genbank Accession #P27539) and human GDF1 (SEQ ID NO:41, Genbank Accession #NP_{—}001483).

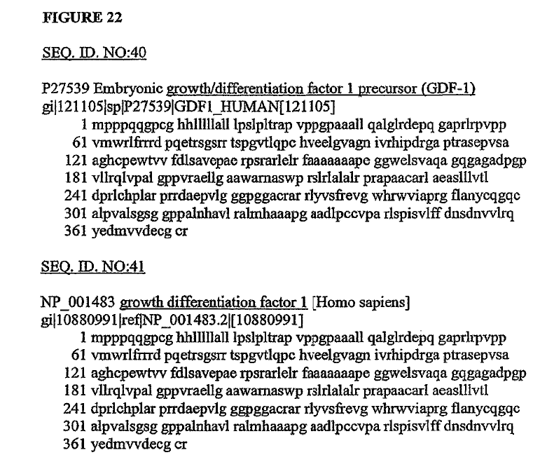

FIG. 23 shows the amino acid sequence of human GDF3 precursor (SEQ ID NO:42, Genbank Accession #NP_{—}065685 and SEQ ID NO:43, Genbank Accession #Q9NR23).

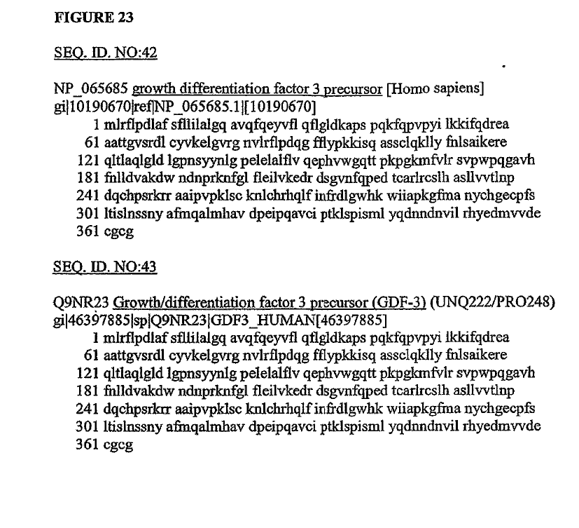

FIG. 24 shows the amino acid sequence of human GDF5 precursor (SEQ ID NO:44, Genbank Accession #P43026) and human GDF5n preprotein (SEQ ID NO:45, Genbank Accession #NP_{—}000548).

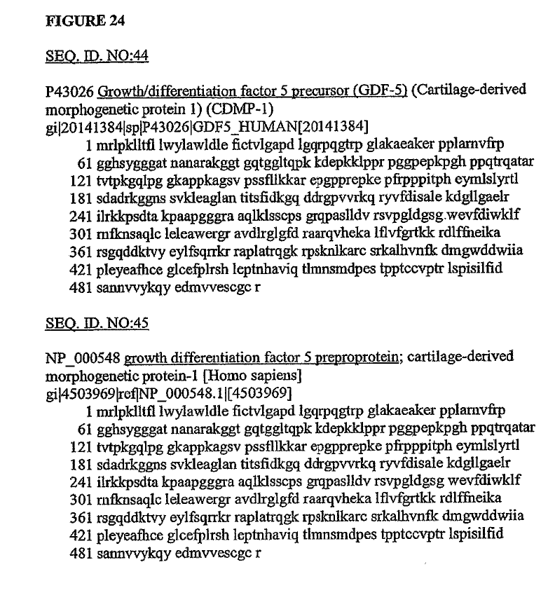

FIG. 25 shows the amino acid sequence of bovine GDF6 precursor (SEQ ID NO:46, Genbank Accession #P55106) and human GDF8 precursor (SEQ ID NO:47, Genbank Accession #O14793).

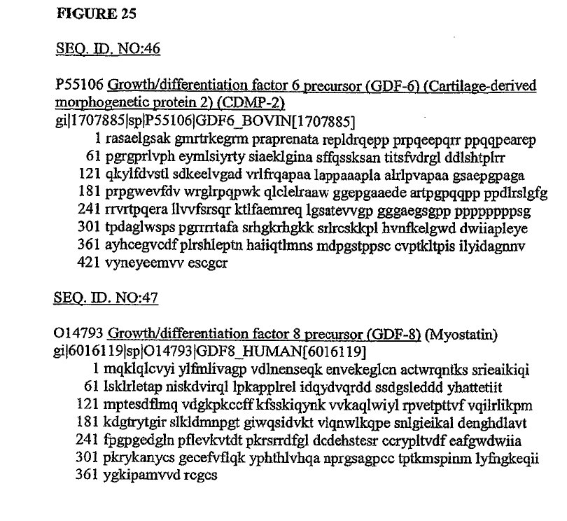

FIG. 26 shows the amino acid sequence of human GDF8 (SEQ ID NO:48, Genbank Accession #NP_{—}005250) and human GDF9 precursor (SEQ ID NO:49, Genbank Accession #NP_{—}005251).

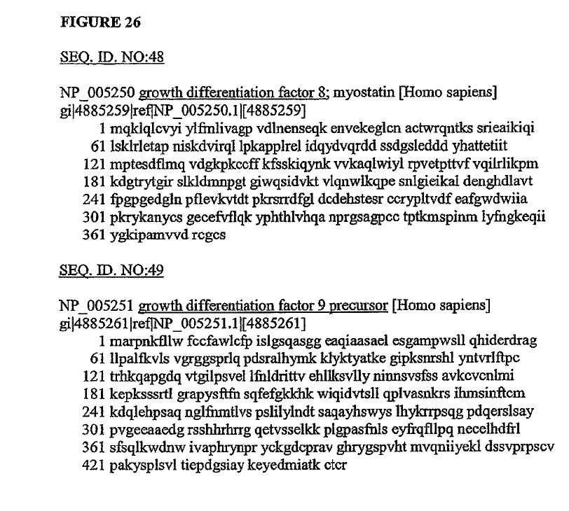

FIG. 27 shows the amino acid sequence of human GDF10 (SEQ ID NO:50, Genbank Accession #AAH28237) and human GDF15 precursor (SEQ ID NO:51, Genbank Accession #Q99988).

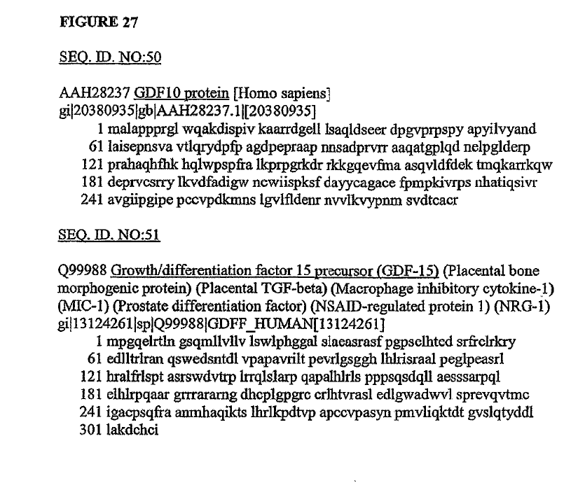

FIG. 28 shows the amino acid sequence of human GDF15 (SEQ ID NO:52, Genbank Accession #NP_{—}004855) and TGFβ (SEQ ID NO:53, Genbank Accession #AAA36738).

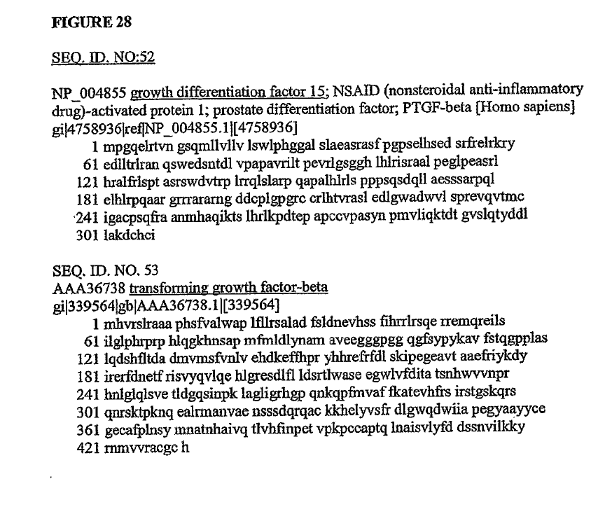

FIG. 29 shows the amino acid sequence of human TGFβ1 (SEQ ID NO:54, GenBank Accession #AAL27646, and SEQ ID NO:55, GenBank Accession #NP_{—}000651).

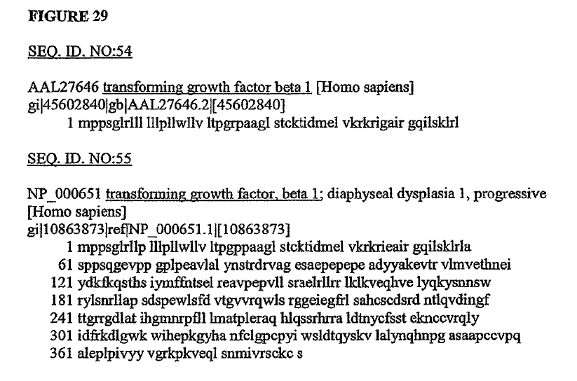

FIG. 30 shows the amino acid sequence of human TGFβ2 precursor (SEQ ID NO: 56, GenBank Accession #P61812, and SEQ ID NO:57, GenBank Accession #AAA50404).

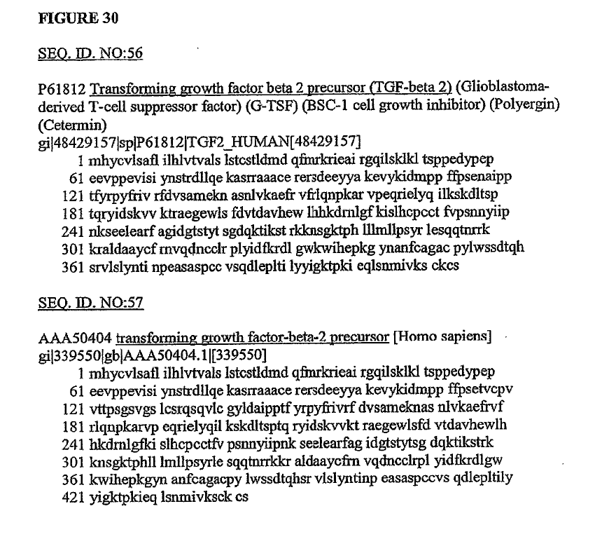

FIG. 31 shows the amino acid sequence for human TGFβ2 (SEQ ID NO:58, GenBank Accession #AAA50405, and SEQ ID NO:59, GenBank Accession #NP_{—}003229).

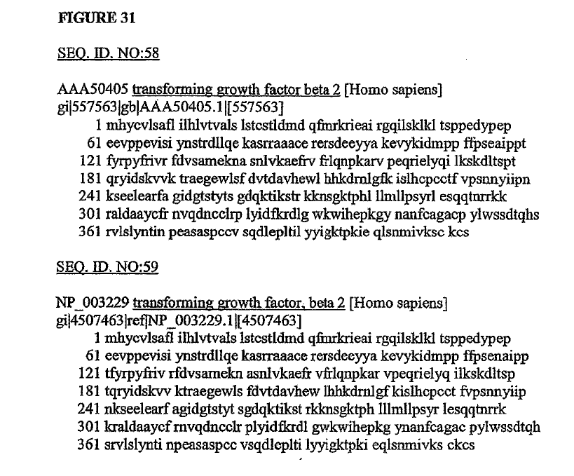

FIG. 32 shows the amino acid sequence of human TGFβ3 precursor (SEQ ID NO:60, GenBank Accession #P10600) and human TGFβ3 (SEQ ID NO:61, GenBank Accession #AAH18503).

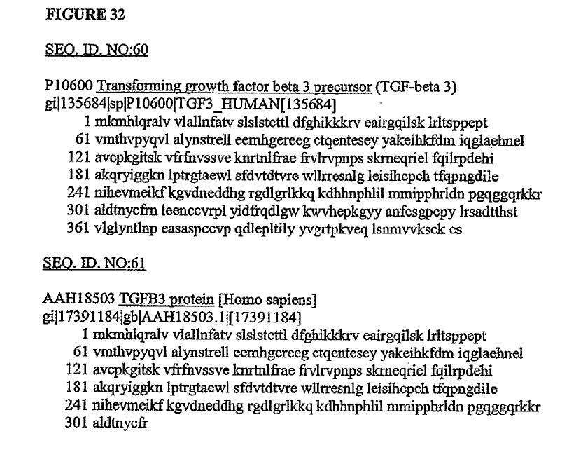

FIG. 33 shows the amino acid sequence of human TGFβ3 (SEQ ID NO:62, GenBank Accession #CAA33024, and SEQ ID NO:63, GenBank Accession #AAC79727).

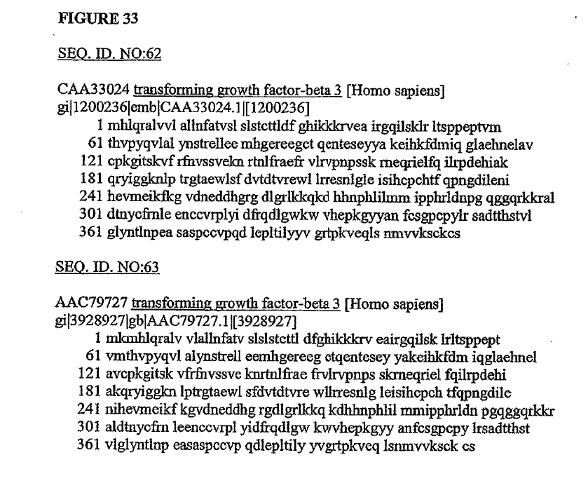

FIG. 34 shows the amino acid sequence of human TGFβ3 (SEQ ID NO:64, GenBank Accession #NP_{—}003230).

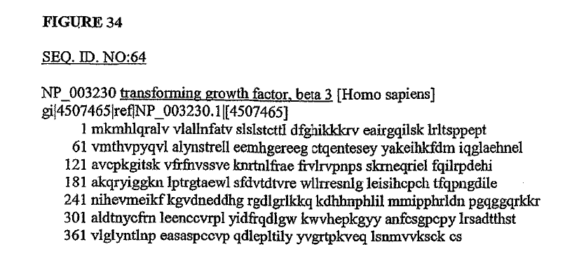

FIG. 35 shows the results of a reverse transcriptase PCR (RT-PCR) two days after transfection of A549 epithelial cells. A 2.56 kb band, the expected size for a BMP 2/7 fusion gene, was detected in the cells transfected with pSCMV-BMP 2/7 (Lane 2), but not in cells transfected with pCMV-GFP (Lane 3) or medium-only control (Lane 4). Neither BMP2 cDNA transcripts alone (expected size 1.2 kb) nor BMP7 cDNA transcripts alone (expected size 1.4 kb) were detected in the pSCMV-BMP2/7 transfected cells.

FIG. 36 shows the results of Western blotting using anti-BMP2 antibody. The majority of mature BMP peptides in supernatants from cells transfected with pSCMV-BMP-2/7 migrated as an immunoreactive band at approximately 39 kDa under non-reducing conditions. This is the expected size for a peptide composed of BMP2, linker and BMP7 (Lane 1). Under reducing conditions, some mature peptides migrated further as products with molecular masses between approximately 15 to approximately 18 kDa (Lane 1A). A similar pattern of migration of mature BMP peptides (at approximately 39 kDa and between approximately 15 kDa to approximately 18 kDa) was detected by anti-BMP2 antibody in supernatants from cells transfected with pSCMV-BMP2/7 that had been immunoprecipitated with either anti-BMP7 antibody (Lane 4) or anti-BMP2 antibody (Lane 6) prior to Western blotting. The “flow-through” portion of the samples collected from the immunoprecipitation columns did not show bands reacting with anti-BMP-2 antibody (Lanes 7-10). A broad band between 45 kDa to 55 kDa was detected in cells transfected with pSCMV-BMP2/7 (Lanes 4 and 6). Western blotting using anti-BMP7 antibody yielded similar results (data not shown).

FIG. 37 is a bar graph which shows that the levels of BMP2 and BMP7 measured by ELISA were similar in the supernatant of cells transfected with pSCMV-BMP2/7. Control supernatants did not contain detectable BMP levels.

FIG. 38 is a bar graph which shows that supernatants of cells transfected with pSCMV-BMP2/7 contained BMP7 as measured by ELISA after immunoprecipitation with anti-BMP2 antibody. Controls did not contain detectable BMP7.

### DETAILED DESCRIPTION

Autologous bone grafting is typically used to provide bone to areas of bone loss in a patient. Such bone loss can occur, for example, as part of a planned orthopedic procedure (e.g., spine fusion surgery) or as a result of trauma (e.g., fractures with avulsed or missing bone). Autologous bone grafting is associated with a high rate of non-union or failure of bone formation (e.g., up to 26% of spine fusion cases) and significant pain at the donor site (usually the iliac crest). Thus, there exists a need for compositions and methods, which result in efficient induction of bone formation in a patient and reduce the pain associated with autologous bone grafting.

BMP homodimers have been used to replace or supplement autologous bone grafting. BMP homodimer treatment has met with some success, but high doses are required and BMP homodimers (e.g., a dimer composed of two BMP2 monomers) are not as potent as BMP heterodimers (e.g., a dimer composed of a BMP2 monomer and a BMP7 monomer). BMP heterodimer treatment is limited by the time, labor and cost consuming process required to produce the heterodimers. Heterodimers are produced by host cells co-transfected with nucleic acids encoding each monomer. The monomers are expressed by the host cells and can dimerize into one of two homodimers (e.g., BMP2 homodimer or a BMP7 homodimer) or a heterodimer (e.g., a BMP-2/7 heterodimer). Because of the similarity between BMP monomers, separation of the heterodimers from the homodimers is difficult.

BMP fusion genes and BMP fusion proteins of the present invention result in more effective treatment for bone loss than BMP monomers because BMP fusion protein is as potent as BMP heterodimer. Expression of the BMP fusion gene results in one polypeptide, which folds to include a functional first BMP protein component and a functional second, different BMP protein component. Because there is no dimerization and no BMP homodimers are formed, BMP fusion protein production avoids the co-transfection and separation steps required for BMP heterodimer production. The present invention also provides methods for treatment of patients with bone loss by administering a BMP fusion protein or BMP fusion gene whereby autologous bone grafting may be replaced or supplemented to provide a higher success rate and, possibly, less patient pain.

### DEFINITIONS

A “BMP fusion gene” as used herein means a nucleotide sequence encoding a BMP fusion protein (e.g., a BMP 2/7 fusion protein). A “human BMP fusion gene” means a BMP fusion gene wherein the nucleotide sequence encoding the first BMP protein component and the nucleotide sequence encoding the second, different BMP protein component are human BMP nucleotide sequences. Preferably, the linker nucleotide sequence is found in the human genome. A “BMP fusion gene” as used herein encompasses a nucleotide sequence encoding a BMP fusion protein, which includes a BMP protein component and a TGF-β superfamily protein component, wherein the TGF-β superfamily protein component is a different protein than the BMP protein component (e.g., a BMP-15/GDF-9 fusion protein).

A “BMP fusion protein” as used herein means a protein, which includes a first BMP protein component, a linker, and a second, different BMP protein component. A “human BMP fusion protein” means a BMP fusion protein wherein the first BMP protein component and the second, different BMP protein component are human BMP protein components. As used herein “BMP fusion protein” encompasses a protein, which includes a BMP protein component, a linker, and a TGF-β superfamily protein component, wherein the TGF-β superfamily protein component is a different protein component than the BMP protein component (e.g., BMP-15/GDF-9 fusion protein).

A “linker” as used herein is (1) a nucleotide sequence within a gene encoding a BMP fusion protein, which encodes an amino acid sequence, and bridges a nucleotide sequence encoding the first BMP protein component and a nucleotide sequence encoding a second, different BMP protein component of the BMP fusion protein, or (2) an amino acid sequence, which bridges the first BMP protein component and the second, different BMP protein component of a BMP fusion protein. A linker according to the present invention can be any nucleotide sequence encoding an amino acid long enough and flexible enough to permit protein folding and not so long as to introduce additional or erroneous folds, extraneous secondary or tertiary folds or other errors to the protein structure. A linker is preferably about 60 bp (about 20 amino acids). An especially preferred linker is (Gly₄Ser)₄ (SEQ ID NO:5).

“Peptidomimetic” as used herein refers to a compound in which at least a portion of the BMP fusion protein is modified, such that the three dimensional structure of the peptidomimetic remains substantially the same as that of the functional BMP protein components of the BMP fusion protein. Alternatively, at least a portion of the BMP fusion protein may be replaced with a nonpeptide structure, such that the three-dimensional structure of the functional BMP protein components of the BMP fusion protein is substantially retained. In addition, other peptide portions of the BMP fusion protein may, but need not, be replaced with a nonpeptide structure. A variety of peptide modifications are known in the art and can be used to generate peptidomimetic compounds. See, for example, International Publication No. WO 01/53331, the contents of which are hereby incorporated in their entirety.

“Subject” or “patient” as used herein means an animal, preferably a mammal, and more preferably a human. Typically a subject or patient is in need of bone formation due to, for example, surgical loss of bone, loss of bone due to traumatic injury, or congenitally missing bone. Additionally, a subject or patient can be any animal, including a laboratory animal in the context of a clinical trial or screening or activity experiment.

The term “therapeutically effective amount” is used herein to mean an amount or dose of a BMP fusion gene or a BMP fusion protein sufficient to induce bone growth in a patient. Alternatively, a therapeutically effective amount of a BMP fusion gene or BMP fusion protein is an amount sufficient to supplement bone formation by known compositions and methods in the art (e.g., autologous bone grafting).

The term Aabout@ or Aapproximately@ means within an acceptable error range for the particular value as determined by one of ordinary skill in the art, which will depend in part on how the value is measured or determined, i.e., the limitations of the measurement system or the degree of precision required for a particular purpose. For example, Aabout@ can mean within 1 or more than 1 standard deviations, per the practice in the art. Alternatively, Aabout@ can mean a range of up to 20%, preferably up to 10%, more preferably up to 5%, and more preferably still up to 1% of a given value.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al (eds.), Current Protocols in Molecular Biology, John Wiley & Sons, Inc. (1994).

“Amplification” of DNA as used herein denotes the use of polymerase chain reaction (PCR) to increase the concentration of a particular DNA sequence within a mixture of DNA sequences. For a description of PCR see Saiki et al, Science 1988, 239:487.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “expression control sequence” refers to a promoter and any enhancer or suppression elements that combine to regulate the transcription of a coding sequence. In a preferred embodiment, the element is an origin of replication.

The terms “vector”, “cloning vector” and “expression vector” refer to the vehicle by which DNA can be introduced into a host cell, resulting in expression of the introduced sequence. In one embodiment, vectors comprise a promoter and one or more control elements (e.g., enhancer elements) that are heterologous to the introduced DNA but are recognized and used by the host cell. In another embodiment, the sequence that is introduced into the vector retains its natural promoter that may be recognized and expressed by the host cell (Bormann et al., J. Bacteriol 1996; 178:1216-1218).

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA, usually of bacterial origin, that can readily accept additional (foreign) DNA and which can be readily introduced into a suitable host cell. A plasmid vector (naked DNA) often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes. Vector constructs may be produced using conventional molecular biology and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); F. M. Ausubel et al. (eds.), Current Protocols in Molecular Biology, John Wiley & Sons, Inc. (1994).

The terms “express” and “expression.” mean allowing or causing the information in a gene or DNA sequence to become manifest for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell.

The terms “transfection” or “transformation” means the introduction of a nucleic acid into a cell, i.e. an extrinsic or extracellular gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” or “transfected” and is a “transformant” or a “clone.” The DNA or RNA introduced to a host cell can come from any source, including cells of the same genus or species as the host cell, or cells of a different genus or species.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells may be cultured in vitro or in vivo in one or more cells in a non-human animal (e.g., a transgenic animal or a transiently transfected animal). For the present invention, host cells include but are not limited to Streptomyces species, E. coli, and human fibroblasts.

The term “expression system” means a host cell and compatible vector under suitable conditions, e.g. for the expression of a protein coded for by foreign DNA carried by the vector and introduced to the host cell. In a specific embodiment, the host cell of the present invention is a Gram-negative or Gram-positive bacteria. These bacteria include, but are not limited to, E. coli and Streptomyces species. An example of a Streptomyces species that may be used includes, but is not limited to, Streptomyces hygroscopicus. In another embodiment, the host cell is a human fibroblast.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is an element operatively associated with a different gene than the one it is operatively associated with in nature.

The terms “mutant” and “mutation.” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence.

The term “variant” may also be used to indicate a modified or altered gene, DNA sequence, enzyme, cell, etc., i.e., any kind of mutant. Two specific types of variants are “sequence-conservative variants”, a polynucleotide sequence where a change of one or more nucleotides in a given codon position results in no alteration in the amino acid encoded at that position, and “function-conservative variants”, where a given amino acid residue in a protein or enzyme has been changed without altering the overall conformation and function of the polypeptide. Amino acids with similar properties are well known in the art. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Clustal Method, wherein similarity is based on the algorithms available in MEGALIGN. A “function-conservative variant” also includes a polypeptide or enzyme which has at least 60% amino acid identity as determined by BLAST or FASTA alignments, preferably at least 75%, more preferably at least 85%, and most preferably at least 90%, and which has the same or substantially similar properties or functions as the native or parent protein or enzyme to which it is compared.

As used herein, the terms “homologous” and “homology” refer to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667, 1987). Such proteins (and their encoding genes) have sequence homology, as reflected by their sequence similarity, whether in terms of percent similarity or the presence of specific residues or motifs at conserved positions.

Accordingly, the term “sequence similarity” refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90% or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al, supra). The conditions of temperature and ionic strength determine the “stringency” of the hybridization. For preliminary screening for homologous nucleic acids, low stringency hybridization conditions, corresponding to a Tₘ (melting temperature) of 55EC, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.1SM NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of T, for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al, supra, 9.50-9.51). For hybridization with shorter nucleic acids, i.e., oligonucleotides, the position of mismatches becomes more important, and the length of the oligonucleotide determines its specificity (see Sambrook et al, supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55EC, and utilizes conditions as set forth above. In a preferred embodiment, the Tₘ is 60EC; in a more preferred embodiment, the Tₘ is 65EC. In a specific embodiment, “high stringency” refers to hybridization and/or washing conditions at 68EC in 0.2×SSC, at 42EC in 50% formamide, 4×SSC, or Linder conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

### TGF-β Gene Superfamily Proteins

TGF-β gene superfamily proteins include for example, BMP proteins, growth and differentiation factor (GDF) proteins, and transforming growth factor proteins (TGF). The active form of most TGF-β proteins contain one or more conserved cysteine residues, which form one or more interchain disulfide bonds resulting in the formation of homodimers. Proteins from different subfamilies of the TGF-β superfamily have been observed to exist as heterodimers. For example, BMP-15 and GDF-9 have been observed. BMP-15 and GDF-9 are closely related in their primary structures and share a nearly identical spatiotemporal expression pattern in the oocyte during folliculogenesis in mammals (Liao et al., J Biol. Chem. 2003; 278(6):3713-9). Several other members of the TGF-β protein family also exist as heterodimers (e.g., inhibin/activin, TGF-β1/TGF-β2) (Israel et al., Growth Factors. 1996; 13(3-4):291-300).

### BMP Proteins

With the exception of BMP-1, BMP proteins are a subgroup of the TGF-β gene superfamily. All BMP proteins, except for BMP-1 form homodimers and several heterodimeric forms have also been described. Examples of BMP heterodimers include BMP-2/3, BMP-2/4, BMP-2/5, BMP-2/6, BMP-2/7, BMP-4/3, BMP-4/5, BMP-4/6, and BMP-4/7 (Israel et al., Growth Factors. 1996; 13:291-300; Suzuki et al., Biochem Biophys Res Commun. 1997 Mar. 6; 232(1):153-6; Aono et al., Biochem Biophys Res Commun. 1995 May 25; 210(3):670-7). BMP proteins also form heterodimers with other TGF-β subfamily proteins. For example, BMP-2/TGF-β1, BMP-4/TGF-β1, BMP-7/GDF-7 and BMP-15/GDF-9 heterodimers have been described (Israel et al., Growth Factors. 1996; 13:291-300; Butler S J and Dodd J, Neuron. 2003; 38(3):389-401; Liao et al., J Biol. Chem. 2003; 278(6):3713-9).

### BMP Fusion Gene

A BMP fusion gene comprises, sequentially, a first full-length BMP gene segment excluding the first BMP stop codon, a linker, and a second, different full-length BMP gene segment excluding the second, different BMP start codon and the nucleotide sequence encoding the second, different BMP signal peptide.

### The First BMP Gene Segment

A BMP fusion gene of the present invention includes a nucleotide sequence encoding a first BMP protein component. The first BMP fusion gene can be DNA or RNA. Nucleic acid encoding the first BMP protein component can be obtained from mRNA present in human osteosarcoma cell lines such as U-2 OS cells or Sa-OS cells. It is also possible to obtain nucleic acid encoding the first BMP from human cell genomic DNA. For example, the gene encoding the first BMP can be cloned from either a cDNA or a genomic library in accordance with standard protocols. A cDNA encoding the first BMP can be obtained by isolating total mRNA from an appropriate cell line, such as U2-OS cells. See, e.g., Example 1. Double stranded cDNAs can then be prepared from the total mRNA. Subsequently, the cDNAs can be inserted into a suitable plasmid or bacteriophage vector using any one of a number of known techniques. Genes encoding the first BMP can also be cloned using established polymerase chain reaction techniques. For example, a DNA vector containing a first BMP cDNA can be used as a template in PCR reactions using oligonucleotide primers designed to amplify a desired region of the first BMP cDNA. In a preferred embodiment, the first BMP fusion gene encodes a BMP-2 protein.

### The Linker

A linker as applied to a BMP fusion gene is a nucleotide sequence within a BMP fusion gene, which encodes an amino acid sequence and bridges a nucleotide sequence encoding the first BMP protein component and the nucleotide sequence encoding the second, different BMP protein component of a BMP fusion protein. A linker according to the present invention can be any nucleotide sequence encoding an amino acid long enough and flexible enough to permit protein folding and not so long as to introduce additional or erroneous folds or other secondary and/or tertiary protein structures. A preferred linker has about 60 bp (encoding about 20 amino acids). An especially preferred linker encodes the amino acid sequence (Gly₄Ser)₄ (SEQ ID NO:5).

In an embodiment of the invention, a linker is a nucleotide sequence present in the human genome such that the likelihood of an immunological reaction upon administration of a BMP fusion gene or a BMP fusion protein is reduced relative to administration of such a gene or protein that did not contain a human genomic linker.

In a particular embodiment, a nucleotide sequence encoding a (Gly₄Ser)₄ (SEQ ID NO:5) linker follows, in order, the start codon of a first BMP gene and the full length of the first BMP gene, excluding the stop codon. The linker is followed by a second, different BMP gene excluding the start codon of the second, different BMP gene and the signal peptide nucleotide sequence of the second, different BMP (i.e., following the linker, the fusion gene encodes the second different BMP gene after the signal peptide up to and including the second, different BMP stop codon).

### The Second, Different BMP Gene Segment

A BMP fusion gene of the invention includes a nucleotide sequence encoding a second, different BMP protein component. The nucleic acids of the second, different BMP fusion gene can be DNA or RNA. Nucleic acid encoding the second different BMP protein component can be obtained from mRNA present in human osteosarcoma cell lines such as U2-OS or Sa-OS, pancreatic adenocarcinoma, normal brain tissue or normal kidney tissue. It is also possible to obtain nucleic acid encoding the second, different BMP protein from human cell genomic DNA. For example, the gene encoding the second, different BMP can be cloned from either a cDNA or a genomic library in accordance with standard protocols. A cDNA encoding the second, different BMP protein can be obtained by isolating total in RNA from an appropriate cell line. See Example 1. Double stranded cDNAs can then prepared from the total mRNA. Subsequently, the cDNAs can be inserted into a suitable plasmid or bacteriophage vector using any one of a number of known techniques. Genes encoding the second, different BMP protein can also be cloned using established polymerase chain reaction techniques. For example, a DNA vector containing the second, different BMP cDNA can be used as a template in PCR reactions using oligonucleotide primers designed to amplify a desired region of the second, different BMP cDNA. In a preferred embodiment, the second, different BMP fusion gene encodes a BMP-7 protein.

It is understood that, according to the present invention, the first or second, different BMP gene segment can be a nucleotide sequence encoding a non-BMP, TGF-β superfamily protein.

### BMP Fusion Protein

A BMP fusion protein of the present invention is more potent for inducing bone formation than a BMP homodimer of either the first or second, different BMP protein. A BMP fusion protein is expressed as one polypeptide, which folds into its functional configuration. Thus, a BMP fusion protein is not a heterodimer. In accordance with the present invention, a BMP fusion protein comprises a first BMP protein component and a second, different BMP protein component. A preferred BMP fusion protein according to the present invention is a BMP-2/7 fusion protein. A preferred linker according to the invention is about 20 amino acids in length. An especially preferred linker is (Gly₄Ser)₄ (SEQ ID NO:5).

It is understood that, according to the present invention, the first BMP protein component or the second, different BMP protein component can be a non-BMP, TGF-β superfamily protein. A BMP fusion protein according to the invention can be any combination of a BMP protein component and a second, different protein component selected from among the TGF-β superfamily proteins wherein the first and second proteins, and an intervening linker, are expressed as one polypeptide such that each protein component folds into its functional conformation post-expression.

In an aspect of the present invention, the BMP fusion protein is modified, and the modified BMP fusion protein comprises addition, removal or substitution of at least one amino acid. These amino acid substitutions include, but are not necessarily limited to, amino acid substitutions known in the art as “conservative”. For example, it is a well-established principle of protein chemistry that certain amino acid substitutions, entitled “conservative amino acid substitutions,” can frequently be made in a protein without altering either the conformation or the function of the protein. Such changes include substituting any of isoleucine (1), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

The present invention provides methods for producing a BMP fusion protein, wherein a host cell transfected with a BMP expression vector is cultured under conditions that provide for expression of the BMP fusion protein. In a further aspect of the invention, a BMP fusion gene is transfected into a eukaryotic cell with a BMP expression vector, the eukaryotic cell is cultured under conditions that provide for the expression of the BMP fusion protein, and the BMP fusion protein produced by the cultured eukaryotic cell is recovered. In another aspect of the invention, a BMP fusion gene is transfected into a prokaryotic cell with a BMP expression vector, the prokaryotic cell is cultured under conditions that provide for the expression of the BMP fusion protein, and the BMP fusion protein produced by the cultured prokaryotic cell is recovered. In preferred embodiments, the present invention provides methods for producing a BMP-2/7 fusion protein, wherein a host cell transfected with a BMP-2/7 expression vector is cultured under conditions that provide for expression of the BMP-2/7 fusion protein.

### Methods for Making a BMP Fusion Gene

A BMP fusion gene construct can be made by methods well known to one of ordinary skill in the art. For example, a BMP fusion gene construct can be made using serial polymerase chain reactions. In one embodiment, a BMP-2/7 fusion gene construct can be made using serial polymerase reactions. See Example 1. Briefly, total RNA can be extracted from cells, which express both a first BMP and a second, different BMP (e.g., U2-OS human osteoblastic cells), and converted to cDNA using reverse transcription. PCR reactions can be performed in the standard manner, wherein a master mix containing cDNA template, primers, NTPs, buffer, MgCl and taq polymerase is prepared. A first round of polymerase chain reactions can be performed using the cDNA derived from U2-OS cells as the template to produce first BMP+linker; and linker+second, different BMP fragments. Primers for first BMP+linker fragment are designed to produce a PCR product in which a KpnI site is added to the 5′ end of the first BMP, and the first BMP stop codon is replaced with a (Gly₄Ser)₄ (SEQ ID NO:5) linker at the 3′ end of the first BMP sequence. Primers for linker+second, different BMP can be designed to produce a PCR product in which the signal peptide sequence at the 5′ end of the second, different BMP is replaced with a (Gly₄Ser)₄ (SEQ ID NO:5) linker and a NotI site is added at the 3′ end of the second, different BMP. A second round of PCR can use the first BMP+linker and linker+second, different BMP PCR products as templates. Each of these fragments is gel purified prior to the second round of PCR. The first BMP+linker and linker+second, different BMP fragments are fused in the second round PCR using the 5′ first BMP (forward) primer and the 3′ second, different BMP (reverse) primer. In this manner, a BMP fusion gene is produced.

### Expression Vectors, Host Cells, and Methods for Producing a BMP Fusion Protein

The BMP fusion protein of the invention can be expressed by incorporating a chimeric BMP fusion gene described herein into an expression vector and introducing the expression vector into an appropriate host cell. Accordingly, the invention further pertains to expression vectors containing a BMP fusion gene and to host cells into which such expression vectors have been introduced.

An expression vector of the invention can be used to transfect cells, either prokaryotic or eukaryotic (e.g., mammalian, insect or yeast cells) to thereby produce fusion proteins encoded by nucleotide sequences of the vector. Expression in prokaryotes is most often carried out in E. coli with vectors containing constitutive or inducible promoters. Certain E. coli expression vectors (so called fusion-vectors) are designed to add a number of amino acid residues to the expressed recombinant protein, usually to the amino terminus of the expressed protein. Such fusion vectors typically serve three purposes: 1) to increase expression of recombinant protein; 2) to increase the solubility of the target recombinant protein; and 3) to aid in the purification of the target recombinant protein by acting as a ligand in affinity purification. Examples of fusion expression vectors include pGEX (Amrad Corp., Melbourne, Australia) and pMAL (New England Biolabs, Beverly, Mass.) which fuse glutathione S-tranferase and maltose E binding protein, respectively, to the target recombinant protein. Accordingly, a BMP fusion gene may be linked to additional coding sequences in a prokaryotic fusion vector to aid in the expression, solubility or purification of the fusion protein. Often, in fusion expression vectors, a proteolytic cleavage site is introduced at the junction of the fusion moiety and the target recombinant protein to enable separation of the target recombinant protein from the fusion moiety subsequent to purification of the fusion protein. Such enzymes, and their cognate recognition sequences, include Factor Xa, thrombin and enterokinase.

Inducible non-fusion expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from the hybrid trp-lac fusion promoter. Target gene expression from the pET 11d vector relies on transcription from the T7 gn10-lac 0 fusion promoter mediated by a coexpressed viral RNA polymerase (T7 gn1). This viral polymerase is supplied by host strains BL21(DE3) or HMS174(DE3) from a resident .lambda. prophage harboring a T7 gn1 under the transcriptional control of the lacUV 5 promoter.

Alternatively, a BMP fusion protein can be expressed in a eukaryotic host cell, such as mammalian cells (e.g., Chinese hamster ovary cells (CHO) or NS0 cells), insect cells (e.g., using a baculovirus vector) or yeast cells. Other suitable host cells are known to those skilled in the art. For expression in mammalian cells, the expression vector's control functions are often provided by viral material. For example, commonly used promoters are derived from polyoma, Adenovirus 2, cytomegalovirus and Simian Virus 40. To express a BMP fusion protein in mammalian cells, generally COS cells (Gluzman, Y., (1981) Cell 23:175-182) are used in conjunction with such vectors as pCDM8 (Seed, B., (1987) Nature 329:840) for transient amplification/expression, while CHO (dhfr.sup.—Chinese Hamster Ovary) cells are used with vectors such as pMT2PC (Kaufman et al. (1987), EMBO J. 6:187-195) for stable amplification/expression in mammalian cells. A preferred cell line for production of recombinant protein is the NS0 myeloma cell line available from the ECACC (catalog #85110503) and described in Galfre, G. and Milstein, C. ((1981) Methods in Enzymology 73(13):3-46; and Preparation of Monoclonal Antibodies: Strategies and Procedures, Academic Press, N.Y., N.Y.). Examples of vectors suitable for expression of recombinant proteins in yeast (e.g., S. cerivisae) include pYepSec1 (Baldari. et al., (1987) Embo J. 6:229-234), pMFa (Kurjan and Herskowitz, (1982) Cell 30:933-943), pJRY88 (Schultz et al., (1.987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell. Biol. 3:2156-2165) and the pVL series (Lucklow, V. A., and Summers, M. D., (1989) Virology 170:31-39).

Vector DNA can be introduced into prokaryotic or eukaryotic cells via conventional transformation or transfection techniques such as calcium phosphate or calcium chloride co-precipitation, DEAE-dextran-mediated transfection, lipofection, or electroporation. Suitable methods for transforming host cells can be found in Sambrook et al. (Molecular Cloning: A Laboratory Manual, 2nd Edition, Cold Spring Harbor Laboratory press (1989)), and other laboratory textbooks.

For stable transfection of mammalian cells, it is known that, depending upon the expression vector and transfection technique used, only a small fraction of cells may integrate DNA into their genomes. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same plasmid as the gene of interest or may be introduced on a separate plasmid. Cells containing the gene of interest can be identified by drug selection (e.g., cells that have incorporated the selectable marker gene will survive, while the other cells die). The surviving cells can then be screened for production of BMP fusion protein by, for example, immunoprecipitation from cell supernatant with an anti-BMP fusion protein monoclonal antibody.

The invention also features methods for producing BMP fusion protein. For example, a host cell transfected with a nucleic acid vector directing expression of a BMP fusion gene can be cultured in a medium under appropriate conditions to allow expression of the protein to occur. Suitable mediums for cell culture are well known in the art. Protein can be isolated from cell culture medium, host cells, or both using techniques known in the art for purifying proteins. In a one embodiment, the invention features methods for producing BMP-2/7 fusion protein.

### Methods for Treatment

A BMP fusion protein or BMP fusion gene of the invention can be incorporated into compositions suitable for administration to patients to induce bone formation for the treatment of bone loss. Administration of a BMP fusion protein or BMP fusion gene as described herein can be in any pharmacological form including a therapeutically effective amount of BMP fusion protein or BMP fusion gene and a pharmaceutically acceptable carrier. In an aspect of the present invention, a BMP-2/7 fusion protein or a BMP-2/7 fusion gene can be incorporated into compositions suitable for administration to patients to induce bone formation for the treatment of bone loss.

The active compound (e.g., BMP fusion protein or BMP fusion gene) can be administered in a convenient manner such as by topical application (i.e., direct application to the bone or to the vicinity of the bone), injection (intra-osseous, intra-articular, subcutaneous, intravenous, etc.), oral administration, inhalation, transdermal application, or rectal administration. Depending on the route of administration, the active compound may be coated in a material to protect the compound from the action of enzymes, acids and other natural conditions which may inactivate the compound. Preferred routes of administration are topical (i.e., direct application to the bone or to the vicinity of the bone), percutaneous (e.g., intra-osseous or intra-articular injection), and intravenous. Systemic (e.g., intravenous) BMP fusion gene therapy may be targeted to the site of bone loss as by, for example, targeting osteoblasts using an osteoblast-specific promoter. For example, a viral vector containing a BMP-2/7 fusion gene can be modified such that gene expression is regulated and replication of the viral vector is restricted to cells capable of activating an osteocalcin promoter (Kubo et al., Hum Gene Ther. 2003; 14(3):227-41; Hsieh et al., Cancer Res. 2002; 62(11):3084-92).

In one embodiment, a BMP fusion protein such as BMP-2/7 fusion protein or BMP-2/7 fusion gene such as BMP-217 is administered topically (i.e., direct application to the bone or to the vicinity of the bone). The topical route of administration is advantageous because the site of bone loss requiring treatment is frequently exposed as a result of surgery or trauma and, thus, the BMP fusion protein or Bmp fusion gene can be directly applied to the target site. Pharmaceutically acceptable carriers especially suited for topical administration (i.e., direct application to the bone or to the vicinity of the bone) include gelatin hemostasis sponge (Gelfoam), Type I collagen gel, deactivated demineralized bone matrix, and any carrier used for the topical delivery of rhBMP2 or 7. For example, an area of bone loss can be associated with bleeding from cut bone edges. A BMP fusion protein in the form of a paste or gel can be applied to a gelatin hemostasis sponge, which is applied to the area of bleeding and bone loss. In this manner, the patient can be treated for both bone loss and the bleeding. In one embodiment of the invention, the BMP fusion protein or BMP fusion gene is delivered directly to the site of bone loss in combination with a matrix providing a structure for developing bone. Matrix material can include, for example, calcium sulfate, tricalciumphosphate, hydroxapatite, polylactic acid, polyglycolic acid, polyanhydrides, and mixtures thereof. According to the present invention, BMP fusion protein or BMP fusion gene can be co-administered with autograft or allograft bone.

Generally, topical administration (i.e., direct application to the bone or to the vicinity of the bone) of a BMP fusion gene or BMP fusion protein will be a one-time application. Typically, the one-time topical administration will be applied prior to closure of a traumatic wound or other defect that has resulted in exposed bone (e.g., a patient with an open bone fracture is administered a BMP fusion protein directly to the fracture site in an emergency room), or at the time of surgery. In some circumstances, repeat topical dosing can be administered such as, for example, during re-operation for non-healing bone.

A BMP fusion protein or BMP fusion gene can be in the form of a powder. A BMP fusion protein powder can be produced, for example, from the supernatant of a producer cell line (mammalian or bacterial) genetically modified to overexpress BMP fusion protein, which is purified and lyophilized. In one embodiment, the BMP fusion protein powder can be wetted to form a gel or paste for topical administration (e.g., placement into the bone gap of a fracture).

Pharmaceutical compositions suitable for injectable use include sterile aqueous solutions or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the BMP fusion protein or BMP fusion gene in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, preferred methods of preparation include vacuum drying and freeze-drying which yields a powder of the active ingredient (e.g., peptide) plus any additional desired ingredient from a previously sterile-filtered solution thereof.

A vector according to the present invention can be delivered in vivo or ex vivo. In vivo delivery of a BMP fusion gene vector means that the BMP fusion gene and a pharmaceutically acceptable carrier are administered directly to the patient. Ex vivo delivery of a BMP fusion gene means that cells from the patient are transfected with the BMP vector in vitro and then the transfected cells are administered to the patient. Suitable cells for ex vivo delivery include, for example, primary marrow stromal cells, muscle stem cells, bone marrow stem cells, chondrocytes, dermal fibroblasts, and gingival fibroblasts.

Any technology suitable for delivery of a therapeutic gene, whether in the form of naked (plasmid) DNA or other vector, is applicable to the BMP fusion gene of the present invention. A liquid suspension dosage form is especially preferred for plasmid or other vector BMP fusion gene therapy.

Administration of a therapeutically active amount of the therapeutic compositions of the invention is defined as an amount effective, at dosages and for periods of time necessary to achieve the desired result. For example, a therapeutically active amount of a BMP fusion protein or BMP fusion gene may vary according to factors such as the disease state, age, sex, and weight of the individual, and the ability of protein to elicit a desired response in the individual. Dosage regimen may be adjusted to provide the optimum therapeutic response. For example, several divided doses may be administered daily or the dose may be proportionally reduced as indicated by the exigencies of the therapeutic situation. Typically, the effects of a BMP fusion gene or BMP fusion protein would be expected to last for at least 4 weeks. Thus, in a preferred embodiment, a dose of a BMP fusion protein or BMP fusion gene is administered about every four weeks until adequate bone has formed.

To administer a BMP fusion protein or BMP fusion gene by other than parenteral administration, it may be necessary to coat the protein with, or co-administer the protein with, a material to prevent its inactivation. For example, a BMP fusion protein or BMP fusion gene may be administered to an individual in an appropriate carrier, diluent or adjuvant, co-administered with enzyme inhibitors or in an appropriate carrier such as liposomes. Pharmaceutically acceptable diluents include saline and aqueous buffer solutions. When the BMP fusion protein or BMP fusion gene is suitably protected, as described above, the protein may be orally administered, for example, with an inert diluent or an assimilable edible carrier.

It is especially advantageous to formulate parenteral and topical compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutically acceptable carrier.

Patents, patent applications, publications, product descriptions, and protocols are cited throughout this application, the disclosures of which are incorporated herein by reference in their entireties for all purposes.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope.

### Example 1

### Construction of cDNA Encoding a BMP2/7 Fusion Protein Using Serial Polymerase Chain Reactions

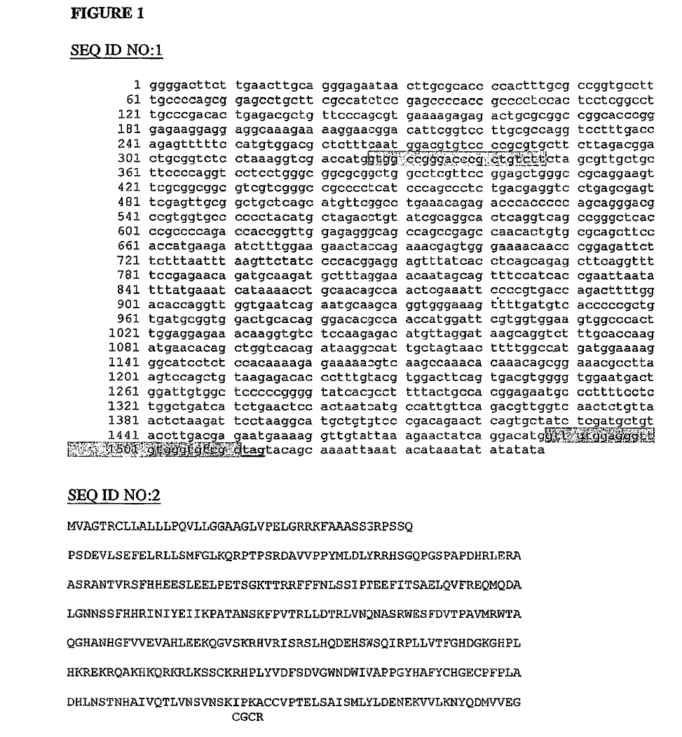

The 5′ end of a gene encoding a BMP-2/7 fusion protein begins with the start codon of BMP2 and continues through the full length of the BMP2 gene, excluding the stop codon. The BMP2 nucleotide sequence is followed by a (Gly₄Ser)₄ (SEQ ID NO:5) linker, which replaces the stop codon of the BMP2 gene, the start codon of the BMP7 gene, and the signal peptide nucleotide sequence of BMP7. Following the linker, the fusion gene encodes the remainder of the BMP7 gene (i.e., the BMP7 gene after the signal peptide) up to and including the BMP7 stop codon. See FIG. 1.

Using Trizol reagent (Sigma) according to the manufacturer's instructions, total RNA was extracted from U2-OS human osteoblastic cells, which express both BMP2 and BMP7. Reverse transcription was performed to convert the RNA to cDNA.

PCR reactions were performed in a standard, well known manner. Briefly, a master mix containing cDNA template, primers, NTPs, buffer, MgCl and taq polymerase was prepared. The PCR reactions were run according to the following protocol:

Thermo-cycling: 94 degrees C. for 3 minutes;

35 cycles of:
94 degrees C. for 30 seconds,
55 degrees C. for 1 minute, and
68 degrees C. for 2 minutes;

68 degrees C. for 5 minutes.

The first round of polymerase chain reactions (PCRs) was performed using cDNA derived from U2-OS cells as the template to produce BMP2+linker, and linker+BMP7 fragments.

Primers for BMP2+linker were designed to produce a PCR product in which a KpnI site was added to the 5′ end of BMP2, and the BMP2 stop codon was replaced with the (Gly₄Ser)₄ (SEQ ID NO:5) linker at the 3′ end of the BMP2 sequence.

Forward primer:

(SEQ ID NO: 6)

GGTACCACCATGGTGGCCGGGACCCGCTGTCTT
Reverse primer:

(SEQ ID NO: 7)

ACTTCCACCTCCACCACTACCACCTCCTCCACTACCTCCACCTCCACTTC
CTCCACCACCGCGACACCCACAACCCTCCACAAC

The expected PCR product BMP2+linker=1.19 kb+60 bp=1.25 kb.

Primers for linker+BMP7 were designed to produce a PCR product in which the signal peptide sequence at the 5′ end of BMP7 was replaced with the (Gly₄Ser)₄ (SEQ ID NO:5) linker and a NotI site was added at the 3′ end of BMP7. Their sequences are:

Forward primer:

(SEQ ID NO: 8)

GGTGGTGGAGGAAGTGGAGGTGGAGGTAGTGGAGGAGGTGGTAGTGGTGG
AGGTGGAAGTGACTTCAGCCTGGACAACGAGGTG
Reverse primer:

(SEQ ID NO: 9)

GCGGCCGCCTAGTGGCAGCCACAGGCCCGGAC

The expected PCR product linker+BMP7=60 bp+1.317 kb=1.377 kb The second round of PCR used the BMP2+linker and linker+BMP7 PCR products as templates. Each of these fragments was gel purified prior to the second round of PCR. The BMP2+linker and linker+BMP7 fragments were fused in the second round PCR using the 5′ BMP2 (forward) primer and the 3′ BMP7 (reverse) primer. Their sequences are:

Forward primer:

(SEQ ID NO: 6)

GGTACCACCATGGTGGCCGGGACCCGCTGTCTT
Reverse primer:

(SEQ ID NO: 9)

GCGGCCGCCTAGTGGCAGCCACAGGCCCGGAC

The expected PCR product (i.e., BMP2+linker+BMP7) was 2.567 Kb.

### Example 2

### The BMP-2/7 Fusion Gene has been Cloned into an Expression Vector and Transfected into a Producer Cell Line

The BMP2+linker+BMP7 PCR product was gel purified and cloned into Topo PCR 2.1 vector (available from Invitrogen). The cDNA sequence was confirmed. The BMP2+linker+BMP7 PCR product cloned into Topo PCR 2.1 vector was subcloned into the pShuttleCMV (available from Stratagene) expression vector. The BMP-2/7 fusion gene expression vector was transfected into a producer cell line (human lung epithelial carcinoma A549 cells). The supernatant of the transfected cells was shown to contain BMP-2/7 fusion protein by immunoprecipitation.

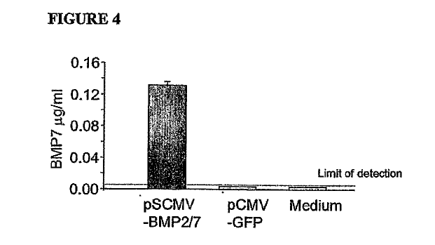

To assess the production of BMP-2/7 fusion protein, pShuttleCMV-BMP-2/7 plasmid DNA was used to transfect A549 cells via a polyfection method (QIAGEN). As controls, A549 cells were transfected with a plasmid encoding the marker gene green fluorescent protein (pCMV-GFP) or no plasmid (“medium”). Forty-eight hours after transfection, supernatants were harvested. To confirm the presence of BMP-2/7 fusion protein, cell supernatants were immunoprecipitated with anti-BMP2 antibody (antibodies available from R&D Systems, Minneapolis, Minn.; Seize X immunoprecipitation kit available from Pierce Biotechnology, Rockford, Ill.) and then by BMP7 ELISA (antibodies available from R&D Systems). Only supernatant of cells transfected with pShuttleCMV-BMP-2/7, which had been immunoprecipitated with anti-BMP2 antibody, contained BMP7. This indicated the presence of BMP-2/7 fusion protein in this group. See FIG. 4. Controls did not contain BMP-2/7 fusion protein. Similar results were observed When cell supernatants were immunoprecipitated with anti-BMP7 antibody and followed by BMP2 ELISA.

These experiments showed that BMP-2/7 fusion gene cloned into an expression vector and transfected into a producer cell line produced BMP-2/7 fusion protein.

### Example 3

### The BMP-2/7 Producer Cell Supernatant was Equipotent to BMP-2/7 Heterodimer Produced by Co-Transfection

BMP stimulation in vitro prevents mouse myoblast C2C12 cells from developing into muscle cells and induces these cells to differentiate into bone-type cells (osteoblasts). C2C12 expression of osteocalcin (OCN), a protein important for matrix mineralization by osteoblasts, was used as a measure of osteoblast differentiation.

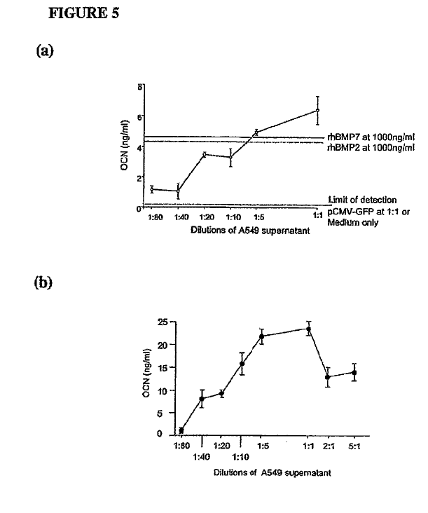

C2C12 cells were stimulated for 7 days with supernatants of A549 producer cells transfected with pShuttleCMV-BMP2/7 plasmid DNA or co-transfected with adenovirus vector encoding BMP2 (AdBMP2) and another adenovirus vector encoding BMP7 (AdBMP7). As controls, A549 cells were transfected with a plasmid encoding the marker gene green fluorescent protein (pCMV-GFP) or no plasmid (“medium”). Supernatants of pShuttleCMV-BMP-2/7 transfected cells containing 5 ng/ml of BMP-2/7 fusion protein (at 1:1 dilution) induced about 6 ng/ml of osteocalcin (OCN) expression. See FIG. 5a. Supernatants generated from co-transfection by BMP2 and BMP7 genes containing 4 ng/ml BMPs (1:80 dilution) or 8 ng/ml BMPs (1:40 dilution) induced about 2 ng/ml and 8 ng/ml of OCN expression in C2C12 cells, respectively. See FIG. 5b.

These experiments showed that BMP-2/7 fusion protein is equipotent to BMP-2/7 heterodimer.

### Example 4

### In Vitro Dose Response Studies Compared BMP-2/7 Heterodimer with rhBMP2 and rhBMP7

OCN concentration was used to measure osteoblast differentiation in C2C12 cells. BMP-2/7 fusion protein administered at a concentration of 2 ng/ml resulted in an OCN level comparable to the OCN level that resulted from administration of 1000 ng/ml of rhBMP2 or rhBMP7. See FIG. 5a.

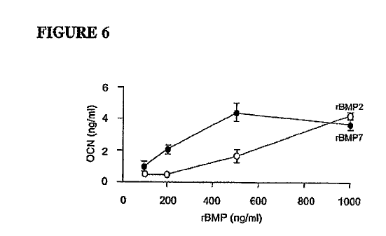

Dose response studies using this C2C12 system also showed that a maximum response to BMP2/7 heterodimer produced by co-transfection of BMP2 and BMP7 genes occurred at a concentration of about 150 ng/ml. See FIG. 5b. This result demonstrated that BMP-2/7 heterodimer induces OCN levels that are about 6-fold higher than can be induced by maximal doses (i.e., 1000 ng/ml) of rhBMP2 or rhBMP7. See FIG. 6.

These results confirmed that BMP-2/7 heterodimer is a more potent inducer of osteoblast differentiation than rhBMP2 or rhBMP7. Thus, a BMP-2/7 fusion protein equipotent to a BMP-2/7 heterodimer should be a more potent inducer of osteoblast differentiation than rhBMP2 or rhBMP7.

### Example 5

### Reverse Transcriptase-PCR (RT-PCR) of Cells Transfected with pSCMV-BMP 2/7 Plasmid

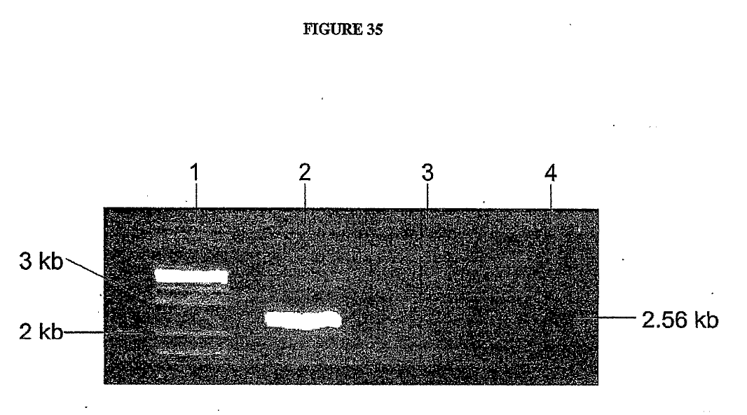

A549 epithelial cells were transfected with pCMV-BMP2/7, PCMV-GFP, or medium-only control. Two days later RT-PCR was performed. FIG. 35. A 2.56 kb band, the expected size for a BMP 2/7 fusion gene, was detected in the cells transfected with pSCMV-BMP 2/7 (Lane 2), but not in cells transfected with pCMV-GFP (Lane 3) or medium-only control (Lane 4). Neither BMP 2 cDNA transcripts alone (expected size 1.2 kb) nor BMP 7 cDNA transcripts alone (expected size 1.4 kb) were detected in pSCMV-BMP2/7 transfected cells. Moreover, BMP2/7 fusion gene was not amplified from RNA of cells transfected with pSCMV-BMP2/7 without the addition of reverse transcriptase. Thus, the amplified BMP2/7 fusion gene product was not contributed directly by plasmid DNA of pSCMV-BMP2/7.

These experiments showed that pSCMV-BMP2/7 transfection resulted in the production of only BMP2/7 fusion gene, and not BMP2 gene alone or BMP7 gene alone.

### Example 6

### The Supernatant of pSCMV-BMP2/7 Transfected Cells Contain BMP2/7 Fusion Protein

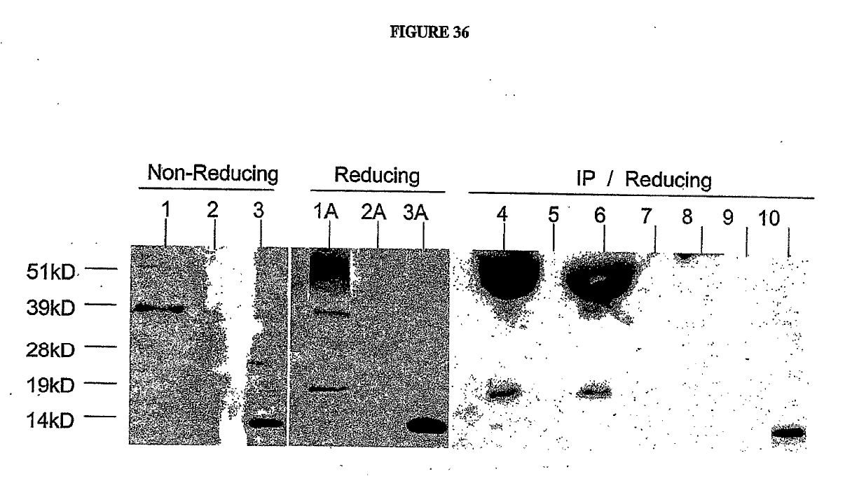

Western blotting was performed on the supernatant of A549 epithelial cells transfected with pSCMV-BMP2/7 using anti-BMP2 antibody. FIG. 36 The majority of mature BMP peptides in the supernatant migrated as an immunoreactive band at approximately 39 kDa under non-reducing conditions. This is the expected size for a peptide composed of BMP2, linker, and BMP7 (Lane 1). B-mecacaptoethanol was added and, under reducing conditions, some mature peptides migrated further as products with molecular masses between approximately 15 to approximately 18 kDa (Lane 1A). This suggested that the 39 kDa fusion gene product was separated into monomers. A similar pattern of migration of mature BMP peptides (at approximately 39 kDa, and between approximately 15 kDa to approximately 18 kDa) was detected by anti-BMP2 antibody in supernatants from cells transfected with pSCMV-BMP2/7 that had been immunoprecipitated with either anti-BMP7 antibody (Lane 4) or anti-BMP2 antibody (Lane 6) prior to Western blotting. The “flow-through” portion of the samples collected from the immunoprecipitation columns did not show bands reacting with anti-BMP-2 antibody (Lanes 7-10). A broad band between 45 kDa to 55 kDa was detected in cells transfected with pSCMV-BMP2/7 (Lanes 4 and 6). This finding indicated the presence of pro-forms of BMP2/7 protein and was consistent with previous studies which found that BMP2 and BMP7 are processed as pro-peptides during protein synthesis. Western blotting using anti-BMP7 antibody yielded similar results (data not shown), which indicated that BMP2/7 fusion protein, but not BMP7 homodimer, had been produced by the BMP2/7 fusion gene-transfected cells.

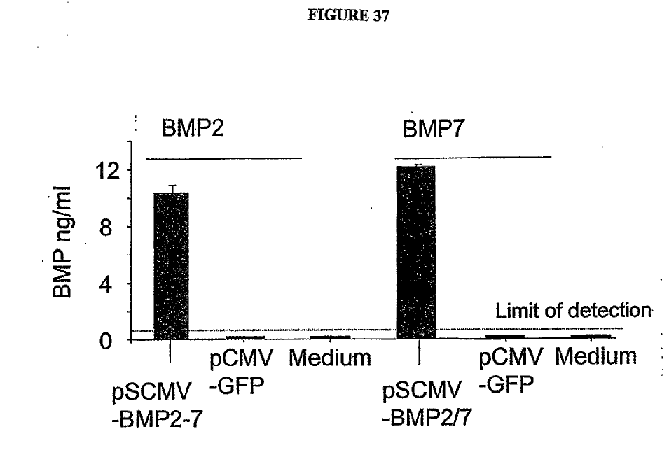

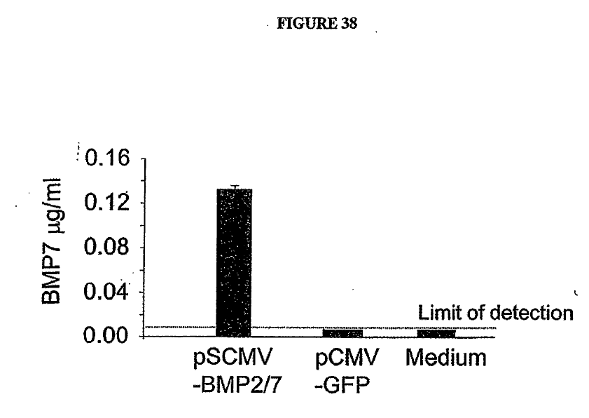

The levels of BMP2 and BMP7 measured by ELISA were similar in the supernatants of cells transfected with pSCMV-BMP2/7. FIG. 37 Control supernatants did not contain detectable BMP levels. After immunoprecipitation with anti-BMP2 antibody, the supernatants of cells transfected with pSCMV-BMP2/7 contained BMP7 as measured by ELISA. FIG. 38 Controls did not contain detectable BMP7.

1. An BMP fusion gene.

2. The BMP fusion gene according to claim 1, wherein the BMP fusion gene is a BMP-2 fusion gene.

3. The BMP fission gene according to claim 2, wherein the BMP fusion gene is a human BMP-2/7 fusion gene.

4. The BMP fission gene of claim 1, which comprises a nucleotide sequence that encodes a first BMP protein, a linker, and a nucleotide sequence that encodes a second, different BMP protein.

5. The BMP-2/7 fusion gene according to claim 4, wherein the nucleotide sequence that encodes a first BMP protein encodes BMP-2 protein, a linker, and the nucleotide sequence that encodes a second, different encodes BMP-7 protein.

6. The BMP fission gene according to claim 4, wherein the linker is a nucleotide sequence having about 60 base pairs.

7. The BMP fusion gene according to claim 6, wherein the linker encodes an amino acid having a sequence (Gly₄Ser)₄ (SEQ ID NO:5).

8. A pharmaceutical composition, which comprises the BMP fusion gene of claim 2 and a pharmaceutically acceptable carrier.

9. A BMP fusion protein.

10. The BMP fusion protein according to claim 9, wherein the BMP fusion protein is a BMP-2/7 fusion protein.

11. The BMP fusion protein according to claim 10, wherein the BMP fusion protein is a human BMP-2/7 fusion protein.

12. The human BMP fusion protein of claim 11, wherein the BMP fusion protein comprises:
(a) a human first BMP protein amino acid sequence;
(b) a linker; and
(c) a human second, different BMP protein amino acid sequence.

13. The human BMP fusion protein of claim 12 wherein the linker is (Gly₄Ser)₄ (SEQ ID NO:5).

14. The BMP fusion protein according to claim 9, which comprises:
(a) a first BMP amino acid sequence as set forth in any one of SEQ ID NOS:2, 4 or 10 to 64;
(b) a linker as set forth in SEQ ID NO:5; and
(c) a second, different BMP amino acid sequence as set forth in any one of any one of SEQ ID NOS:2, 4, or 10 to 64;
wherein the BMP amino acid sequence of (a) is different than the BMP amino acid sequence of (b) and either (a) or (b) is a BMP amino acid sequence as set forth in any one of SEQ ID NOS:2, 4 or 10 to 39.

15. The human BMP-2/7 fusion protein of claim 10, wherein the fusion protein comprises:
(a) a human BMP2 amino acid sequence as set forth in SEQ ID NO:2;
(b) a linker as set forth in SEQ ID NO:5; and
(c) a human BMP7 amino acid sequence as set forth in SEQ ID NO:4;
wherein the linker replaces the BMP2 stop codon, the BMP7 start codon, and the BMP7 signal peptide nucleotide sequence.

16. A BMP fusion gene comprising an isolated nucleic acid having a nucleotide sequence selected from the group consisting of:
(a) a nucleic acid sequence that hybridizes to a nucleotide sequence encoding a fusion protein as set forth in claim 10, said hybridization being performed under stringent conditions;
(b) a nucleic acid sequence encoding a polypeptide at least 90% homologous to a fusion protein as set forth in claims 10; and
(c) an isolated nucleic acid fragment having a nucleotide sequence complementary to the nucleotide sequence of (a) or (b).

17. A pharmaceutical composition comprising a BMP fusion protein according to claim 10 and a pharmaceutically acceptable carrier.

18. An expression vector which comprises the BMP fusion gene of claim 4 operatively associated with an expression control sequence.

19. A method for producing a BMP fusion protein which comprises:
(1) fusing a linker to the 3′ end of a first BMP gene; and
(2) fusing a second, different BMP gene to the 3′ end of the linker;
wherein the linker replaces the first BMP gene stop codon; the second, different BMP start codon; and the second, different BMP signal peptide nucleotide sequence.

20. The method according to claim 19, wherein the first BMP gene is a BMP-2 gene and the second, different BMP gene is a BMP-7 gene.

21. A method for inducing bone growth in a patient, which comprises administering a BMP fusion protein to a patient in need thereof.

22. The method according to claim 21, wherein the BMP fusion protein is administered directly to a bone.

23. The method according to claim 21, wherein the BMP fusion protein is administered in the vicinity of a bone.

24. A method for inducing bone growth in a patient, which comprises administering a BMP fusion gene to a patient in need thereof.

25. A host cell comprising the expression vector of claim 18.

26. A method for producing a BMP-2/7 fusion protein, which method comprises isolating the BMP-2/7 fusion protein produced by the host cell of claim 25, wherein the host cell has been cultured under conditions that provide for expression of the BMP-2/7 fusion protein.

27. A BMP fusion gene according to claim 1, further comprising a nucleotide sequence encoding a BMP protein component, a linker, and a nucleotide sequence encoding a TGF-β superfamily protein component, wherein the TGF-β superfamily protein component is different than the BMP protein component.

28. The BMP fusion gene according to claim 27, wherein the BMP fusion gene encodes a BMP fusion protein selected from the group consisting of: BMP-7/GDF-7; BMP-15/GDF-9; BMP-2/TGF-β1 and BMP-4/TGF-β1.

29. A BMP fusion protein according to claim 9, further comprising a BMP protein component, a linker, and a nucleotide sequence encoding a TGF-β superfamily protein component, wherein the TGF-β superfamily protein component is different than the BMP protein component.

30. The BMP fusion protein according to claim 29, wherein the BMP fusion protein is selected from the group consisting of: BMP-7/GDF-7; BMP-15/GDF-9; BMP-2/TGF-β1 and BMP4/TGF-β1.


SEQUENCE LISTING (64 sequences)

SEQ ID NO: 1 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 1878 bp, Homo sapiens
gggcgcagcggggcccgtctgcagcaagtgaccgacggccgggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatg

SEQ ID NO: 4 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 5 - protein, 20 aa, artificial
  linker
GGGGSGGGGSGGGGSGGGGS

SEQ ID NO: 6 - DNA, 33 bp, artificial
  primer
ggtaccaccatggtggccgggacccgctgtctt

SEQ ID NO: 7 - DNA, 84 bp, artificial
  primer
acttccacctccaccactaccacctcctccactacctccacctccacttcctccaccaccgcgacacccacaaccctccacaac

SEQ ID NO: 8 - DNA, 84 bp, artificial
  primer
ggtggtggaggaagtggaggtggaggtagtggaggaggtggtagtggtggaggtggaagtgacttcagcctggacaacgaggtg

SEQ ID NO: 9 - DNA, 32 bp, artificial
  primer
gcggccgcctagtggcagccacaggcccggac

SEQ ID NO: 10 - protein, 472 aa, Homo sapiens
MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQFLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 11 - protein, 472 aa, Homo sapiens; identical to SEQ ID NO: 10

SEQ ID NO: 12 - protein, 478 aa, Homo sapiens
MAHVPARTSPGPGPQLLLLLLPLFLLLLRDVAGSHRAPAWSALPAAADGLQGDRDLQRHPGDAAATLGPSAQDMVAVHMHRLYEKYSRQGARPGGGNTVRSFRARLEVVDQKAVYFFNLTSMQDSEMILTATFHFYSEPPRWPRALEVLCKPRAKNASGRPLPLGPPTRQHLLFRSLSQNTATQGLLRGAMALAPPPRGLWQAKDISPIVKAARRDGELLLSAQLDSEERDPGVPRPSPYAPYILVYANDLAISEPNSVAVTLQRYDPFPAGDPEPRAAPNNSADPRVRRAAQATGPLQDNELPGLDERPPRAHAQHFHKHQLWPSPFRALKPRPGRKDRRKKGQEVFMAASQVLDFDEKTMQKARRKQWDEPRVCSRRYLKVDFADIGWNEWIISPKSFDAYYCAGACEFPMPKIVRPSNHATIQSIVRAVGIIPGIPEPCCVPDKMNSLGVLFLDENRNVVLKVYPNMSVDTCACR

SEQ ID NO: 13 - protein, 478 aa, Homo sapiens; identical to SEQ ID NO: 12

SEQ ID NO: 14 - protein, 478 aa, Homo sapiens; identical to SEQ ID NO: 12

SEQ ID NO: 15 - protein, 478 aa, Homo sapiens; identical to SEQ ID NO: 12

SEQ ID NO: 16 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 17 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 18 - protein, 408 aa, Homo sapiens; identical to SEQ ID NO: 17

SEQ ID NO: 19 - protein, 408 aa, Homo sapiens; identical to SEQ ID NO: 17

SEQ ID NO: 20 - protein, 402 aa, Homo sapiens
MLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 21 - protein, 408 aa, Homo sapiens; identical to SEQ ID NO: 17

SEQ ID NO: 22 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 23 - protein, 454 aa, Homo sapiens; identical to SEQ ID NO: 22

SEQ ID NO: 24 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 25 - protein, 513 aa, Homo sapiens; identical to SEQ ID NO: 24

SEQ ID NO: 26 - protein, 402 aa, Homo sapiens
MTALPGPLWLLGLALCALGGGGPGLRPPPGCPQRRLGARERRDVQREILAVLGLPGRPRPRAPPAASRLPASAPLFMLDLYHAMAGDDDEDGAPAERRLGRADLVMSFVNMVERDRALGHQEPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 27 - protein, 402 aa, Homo sapiens [sequence omitted]

SEQ ID NO: 28 - protein, 429 aa, Homo sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 29 - protein, 429 aa, Homo sapiens; identical to SEQ ID NO: 28

SEQ ID NO: 30 - protein, 424 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 31 - protein, 424 aa, Homo sapiens; identical to SEQ ID NO: 30

SEQ ID NO: 32 - protein, 424 aa, Homo sapiens; identical to SEQ ID NO: 30

SEQ ID NO: 33 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 34 - protein, 407 aa, Homo sapiens; identical to SEQ ID NO: 33

SEQ ID NO: 35 - protein, 407 aa, Homo sapiens; identical to SEQ ID NO: 33

SEQ ID NO: 36 - protein, 392 aa, Homo sapiens
MVLLSILRILFLCELVLFMEHRAQMAEGGQSSIALLAEAPTLPLIEELLEESPGEQPRKPRLLGHSLRYMLELYRRSADSHGHPRENRTIGATMVRLVKPLTNVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTRQADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 37 - protein, 392 aa, Homo sapiens
MVLLSILRILFLCELVLFMEHRAQMAEGGQSSIALLAEAPTLPLIEELLEESPGEQPRKPRLLGHSLRYMLELYRRSADSHGHPRENRTIGATMVRLVKPLTSVARPHRGTWHIQILGFPLRPNRGLYQLVRATVVYRHHLQLTRFNLSCHVEPWVQKNPTNHFPSSEGDSSKPSLMSNAWKEMDITQLVQQRFWNNKGHRILRLRFMCQQQKDSGGLELWHGTSSLDIAFLLLYFNDTHKSIRKAKFLPRGMEEFMERESLLRRTRQADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 38 - protein, 513 aa, Homo sapiens; identical to SEQ ID NO: 24

SEQ ID NO: 39 - protein, 127 aa, Homo sapiens
YQITAYNHTITYAERRRKQYNDAQRCYRIVEKKLPFRIAFSVIGTEEWKTEFHLCIGVCFHFFLKSLKPSNHATIQSIVRAVGVVPGIPEPCCVPEKMSSLSILFLDENRNVVLKVYPNMTVDTCAC

SEQ ID NO: 40 - protein, 372 aa, Homo sapiens
MPPPQQGPCGHHLLLLLALLLPSLPLTRAPVPPGPAAALLQALGLRDEPQGAPRLRPVPPVMWRLFRRRDPQETRSGSRRTSPGVTLQPCHVEELGVAGNIVRHIPDRGAPTRASEPVSAAGHCPEWTVVFDLSAVEPAERPSRARLELRFAAAAAAAPEGGWELSVAQAGQGAGADPGPVLLRQLVPALGPPVRAELLGAAWARNASWPRSLRLALALRPRAPAACARLAEASLLLVTLDPRLCHPLARPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 41 - protein, 372 aa, Homo sapiens; identical to SEQ ID NO: 40

SEQ ID NO: 42 - protein, 364 aa, Homo sapiens
MLRFLPDLAFSFLLILALGQAVQFQEYVFLQFLGLDKAPSPQKFQPVPYILKKIFQDREAAATTGVSRDLCYVKELGVRGNVLRFLPDQGFFLYPKKISQASSCLQKLLYFNLSAIKEREQLTLAQLGLDLGPNSYYNLGPELELALFLVQEPHVWGQTTPKPGKMFVLRSVPWPQGAVHFNLLDVAKDWNDNPRKNFGLFLEILVKEDRDSGVNFQPEDTCARLRCSLHASLLVVTLNPDQCHPSRKRRAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 43 - protein, 364 aa, Homo sapiens; identical to SEQ ID NO: 42

SEQ ID NO: 44 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 45 - protein, 501 aa, Homo sapiens; identical to SEQ ID NO: 44

SEQ ID NO: 46 - protein, 436 aa, Homo sapiens
RASAELGSAKGMRTRKEGRMPRAPRENATAREPLDRQEPPPRPQEEPQRRPPQQPEAREPPGRGPRLVPHEYMLSIYRTYSIAEKLGINASFFQSSKSANTITSFVDRGLDDLSHTPLRRQKYLFDVSTLSDKEELVGADVRLFRQAPAALAPPAAAPLAALRLPVAPAAGSAEPGPAGAPRPGWEVFDVWRGLRPQPWKQLCLELRAAWGGEPGAAEDEARTPGPQQPPPPDLRSLGFGRRVRTPQERALLVVFSRSQRKTLFAEMREQLGSATEVVGPGGGAEGSGPPPPPPPPPPSGTPDAGLWSPSPGRRRRTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYNEYEEMVVESCGCR

SEQ ID NO: 47 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 48 - protein, 375 aa, Homo sapiens; identical to SEQ ID NO: 47

SEQ ID NO: 49 - protein, 454 aa, Homo sapiens
MARPNKFLLWFCCFAWLCFPISLGSQASGGEAQIAASAELESGAMPWSLLQHIDERDRAGLLPALFKVLSVGRGGSPRLQPDSRALHYMKKLYKTYATKEGIPKSNRSHLYNTVRLFTPCTRHKQAPGDQVTGILPSVELLFNLDRITTVEHLLKSVLLYNINNSVSFSSAVKCVCNLMIKEPKSSSRTLGRAPYSFTFNSQFEFGKKHKWIQIDVTSLLQPLVASNKRSIHMSINFTCMKDQLEHPSAQNGLFNMTLVSPSLILYLNDTSAQAYHSWYSLHYKRRPSQGPDQERSLSAYPVGEEAAEDGRSSHHRHRRGQETVSSELKKPLGPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 50 - protein, 288 aa, Homo sapiens
MALAPPPRGLWQAKDISPIVKAARRDGELLLSAQLDSEERDPGVPRPSPYAPYILVYANDLAISEPNSVAVTLQRYDPFPAGDPEPRAAPNNSADPRVRRAAQATGPLQDNELPGLDERPPRAHAQHFHKHQLWPSPFRALKPRPGRKDRRKKGQEVFMAASQVLDFDEKTMQKARRKQWDEPRVCSRRYLKVDFADIGWNEWIISPKSFDAYYCAGACEFPMPKIVRPSNHATIQSIVRAVGIIPGIPEPCCVPDKMNSLGVLFLDENRNVVLKVYPNMSVDTCACR

SEQ ID NO: 51 - protein, 308 aa, Homo sapiens [sequence omitted]

SEQ ID NO: 52 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDDCPLGPGRCCRLHTVRASLEDLGWADWVLSPREVQVTMCIGACPSQFRAANMHAQIKTSLHRLKPDTEPAPCCVPASYNPMVLIQKTDTGVSLQTYDDLLAKDCHCI

SEQ ID NO: 53 - protein, 431 aa, Homo sapiens; identical to SEQ ID NO: 4

SEQ ID NO: 54 - protein, 59 aa, Homo sapiens
MPPSGLRLLLLLLPLLWLLVLTPGRPAAGLSTCKTIDMELVKRKRIGAIRGQILSKLRL

SEQ ID NO: 55 - protein, 391 aa, Homo sapiens
MPPSGLRLLPLLLPLLWLLVLTPGPPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 56 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 57 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 58 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 59 - protein, 414 aa, Homo sapiens; identical to SEQ ID NO: 56

SEQ ID NO: 60 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 61 - protein, 309 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 62 - protein, 410 aa, Homo sapiens
MHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 63 - protein, 412 aa, Homo sapiens; identical to SEQ ID NO: 60

SEQ ID NO: 64 - protein, 412 aa, Homo sapiens; identical to SEQ ID NO: 60